(19) United States
(12) Patent Application Publication
(10) Pub. No.: US 2025/0082290 A1
(43) Pub. Date: Mar. 13, 2025

(54) IMAGING SYSTEMS AND METHODS, AND DUAL-SOURCE SCANNING SYSTEMS AND METHODS THEREOF

(71) Applicant: SHANGHAI UNITED IMAGING HEALTHCARE CO., LTD., Shanghai (CN)

(72) Inventors: Wenjing CAO, Shanghai (CN); Siyuan ZHANG, Shanghai (CN); Rimao ZOU, Shanghai (CN)

(73) Assignee: SHANGHAI UNITED IMAGING HEALTHCARE CO., LTD., Shanghai (CN)

(21) Appl. No.: 18/827,746

(22) Filed: Sep. 7, 2024

(30) Foreign Application Priority Data
Sep. 7, 2023 (CN) 202311157597.6
Oct. 30, 2023 (CN) 202311422593.6

(51) Int. Cl. A61B 6/00; A61B 6/40; G06T 11/00

(52) U.S. Cl. CPC A61B 6/5205; A61B 6/4014; A61B 6/5258; G06T 11/005; G06T 2210/41

(57) ABSTRACT

The embodiments of the present disclosure provide an imaging method, an imaging system, a dual-source scanning system, and a method for controlling the dual-source scanning system. The imaging method includes: obtaining first detection data and second detection data acquired by scanning an object using a first detector and a second detector of an imaging device respectively, and obtaining a first reconstructed image by performing image reconstruction based on the first detection data and the second detection data. The first reconstructed image can cover an area not detected by the first detector, thus expanding a reconstruction range. The dual-source scanning system includes afirst scanning system and a I second scanning system, wherein at least one of the first detector or the second detector has an asymmetric structure relative to a central channel thereof. A first scanning FOV of the first scanning system and a second scanning FOV of the second scanning system are at least partially overlapped.

### CROSS-REFERENCE TO RELATED APPLICATIONS

This application claims priority to Chinese Patent Application No. 202311157597.6, filed on Sep. 7, 2023, and Chinese Patent Application No. 202311422593.6, filed on Oct. 30, 2023, the entire contents of each of which are incorporated herein by reference.

### TECHNICAL FIELD

The present disclosure relates to the field of medical imaging technology, and in particular to imaging methods and systems, and dual-source scanning systems and methods thereof.

### BACKGROUND

A dual-source scanning device has two sets of X-ray sources and two detector systems. Because dual-source scanning device is limited by reasonable geometric design and gantry space, the two detector systems usually have different fields of view (FOV), with one of the detector systems having a relatively large scanning FOV and the other having a relatively small scanning FOV. In clinical scanning and applications, image reconstruction is greatly limited by the relatively small FOV. If a size of a scanned object exceeds the scanning field of the detector system with the relatively small FOV, it is not possible to accurately reconstruct an image outside the scanning field of the detector system with the relatively small FOV, which leads to problems such as inaccurate reconstructed images and insufficient data.

Accordingly, it is desirable to provide imaging methods and systems, and dual-source scanning systems and methods thereof, which are capable of effectively improving reconstruction accuracy and expanding a reconstruction range.

### SUMMARY

Some embodiments of the present disclosure provide a method for imaging. The method may be implemented on at least one machine each of which has at least one processor and a storage device, and the method may include: obtaining first detection data and second detection data acquired by scanning an object using a first detector and a second detector of an imaging device respectively, wherein field angles on two sides of a central channel of the first detector may be unequal, and a first scanning FOV of the first detector and a second scanning FOV of the second detector are at least partially overlapped; and obtaining a first reconstructed image by performing image reconstruction based on the first detection data and the second detection data.

In some embodiments, the first scanning FOV of the first detector includes at least a part of a first detection area and at least a part of a second detection area, the second scanning FOV of the second detector includes at least a part of the first detection area and at least a part of the second detection area, the first detection area is a circular area, the second detection area is an annular area, and the first detection area being surrounded by the second detection area; ray beams that are emitted from a first radiation source corresponding to the first detector and received by an outermost detector module on one outermost side of the first detector are tangent to the first detection area, ray beams that are emitted from the first radiation source corresponding to the first detector and received by an outermost detector module on another outermost side of the first detector are tangent to an outer ring of the second detection area; ray beams that are emitted from a second radiation source corresponding to the second detector and received by outermost detector modules on two outermost sides of the second detector are tangent to or beyond the second detection area; and a coverage corresponding to the first reconstructed image includes the first detection area and the second detection area.

In some embodiments, the obtaining a first reconstructed image by performing image reconstruction based on the first detection data and the second detection data may include: obtaining extended first detection data by extending the first detection data based on the second detection data, or based on the second detection data and the first detection data; and obtaining the first reconstructed image by performing image reconstruction based on the extended first detection data.

In some embodiments, the obtaining extended first detection data by extending the first detection data based on the second detection data and the first detection data may include: determining virtual detection data for an area to be extended based on the first detection data and the second detection data, wherein the area to be extended is an area detected by the second detector but not detected by the first detector; and obtaining the extended first detection data based on the virtual detection data and the first detection data.

In some embodiments, the second detection data may include first data and second data. The first data may be obtained when a projection angle of the imaging device is at a first preset angle, the second data may be obtained when the projection angle of the imaging device is at a second preset angle, and the first detection data may include third data obtained when the projection angle of the imaging device is at the second preset angle. The first preset angle and the second preset angle may be conjugate.

In some embodiments, the determining virtual detection data for an area to be extended based on the first detection data and the second detection data may include: determining the virtual detection data for the area to be extended based on the first data and difference data between the second data and the third data.

In some embodiments, the first detection data may further include fourth data obtained when the projection angle of the imaging device is at the first preset angle, and the obtaining the first reconstructed image by performing image reconstruction based on the virtual detection data and the first detection data may include: generating composite data corresponding to an area covering the second detection area based on the virtual detection data and the fourth data; and obtaining the first reconstructed image by performing image reconstruction based on the composite data.

In some embodiments, the method may further include: obtaining a second reconstructed image by performing image reconstruction based on the second detection data; and generating a target reconstructed image of the object based on the first reconstructed image and the second reconstructed image.

In some embodiments, the method may further include: obtaining a corrected first reconstructed image by performing reconstruction based on the target reconstructed image and the first detection data; and generating a corrected target reconstructed image of the object based on the second reconstructed image and the corrected first reconstructed image.

In some embodiments, the method may further include: determining a structural similarity between the first reconstructed image and the second reconstructed image; and generating a corrected target reconstructed image of the object by correcting the first reconstructed image based on the structural similarity between the first reconstructed image and the second reconstructed image.

Some embodiments of the present disclosure provide a dual-source scanning system. The dual-source scanning system may include a first detector and a second detector, wherein: at least one of the first detector or the second detector has an asymmetric structure relative to a central channel thereof, and a first scanning field of view (FOV) of the first detector and a second scanning FOV of the second detector are at least partially overlapped.

In some embodiments, the first detector has an asymmetric structure relative to a first central channel of the first detector, the first scanning FOV of the first detector includes a first detection area and at least a part of a second detection area, the second scanning FOV of the second detector includes the first detection area and the second detection area, the first detection area is surrounded by the second detection area, and the first scanning FOV of the first detector is smaller than the second scanning FOV of the second detector.

In some embodiments, the first detector has an asymmetric structure relative to a first central channel of the first detector, the second detector has an asymmetric structure relative to a second central channel of the second detector. The first scanning FOV of the first detector may include at least a part of a first detection area and at least a part of a second detection area, and the second scanning FOV of the second detector includes at least a part of the first detection area and at least a part of the second detection area, the first detection area being surrounded by the second detection area, or the first scanning FOV of the first detector includes at least a part of a third detection area, and the second scanning FOV of the second detector includes at least a part of a fourth detection area, the third detection area and the fourth detection area being partially overlapped.

In some embodiments, the asymmetric structure may be configured as: counts of detector modules on two sides relative to the central channel being different.

In some embodiments, the asymmetric structure may be configured as: arrangement curvatures of detector modules on two sides relative to the central channel being different.

In some embodiments, the asymmetric structure may be configured as: a count of detector modules on a side close to a second focal point relative to the first central channel of the first detector being greater than a count of detector modules on a side away from the second focal point relative to the first central channel of the first detector, the second focal point being the focal point of the second detector.

In some embodiments, the asymmetric structure of the first detector may be configured as: a count of detector modules on a side close to a second focal point relative to the first central channel of the first detector being greater than a count of detector modules on a side away from the second focal point relative to the first central channel of the first detector, the second focal point being the focal point of the second detector; and the asymmetric structure of the second detector is configured as: a count of detector modules on a side close to a first focal point relative to the second central channel of the second detector being greater than a count of detector modules on a side away from the first focal point relative to the second central channel of the second detector, the first focal point being the focal point of the first detector.

In some embodiments, a scan pitch of the dual-source scanning system may be linked to the first scanning FOV of the first detector, and a maximum pitch of the dual-source scanning system is related to at least one of a distance from a focal point of the first detector to the isocenter, a distance from any point in the first scanning FOV to the isocenter, or a count of detector modules of the first detector in a movement direction of a couch of the dual-source scanning system.

Some embodiments of the present disclosure provide a method for controlling a dual-source scanning system. The method may be implemented on at least one machine each of which has at least one processor and a storage device, wherein the dual-source scanning system may include a first detector and a second detector, at least one of the first detector and the second detector has an asymmetric structure relative to a central channel thereof, the first detector has a first scanning field of view (FOV), the second detector has a second scanning FOV, and the first scanning FOV and the second scanning FOV are at least partially overlapped. The method may include: obtaining first detection data acquired by the first detector and second detection data acquired by the second detector by scanning a part of an object to be scanned; and obtaining a target image by performing image reconstruction based on the first detection data and the second detection data.

In some embodiments, the first scanning FOV may include a first detection area and a part of a second detection area, and the second scanning FOV may include the first detection area and the second detection area, the first detection area being surrounded by the second detection area, the generating a target reconstructed image by performing image reconstruction based on the first detection data and the second detection data may include: obtaining extended detection data by performing an extension operation on the first detection data, a scanning FOV corresponding to the extended detection data being larger than the first scanning FOV; and obtaining the target reconstructed image based on the second detection data and the extended detection data.

In some embodiments, the scanning a part of an object to be scanned may include: scanning the part of the object to be scanned using a helical scan; wherein a maximum pitch of the helical scan is related to at least one of a distance from a focal point of the first detector to an isocenter of the dual-source scanning system, a distance from any point in the first scanning FOV to the isocenter, or a count of detector modules of the first detector in the movement direction of the couch.

The beneficial effects brought by some embodiments of the present disclosure may include one or more of the following effects. (1) By setting up an asymmetric first scanning system, the scanning FOV of the first scanning system can be effectively enlarged, and the reconstruction range can be enlarged. (2) The field angles on the two sides of the center channel of the first detector are unequal, and the first scanning FOV of the first detector includes the first detection area and a part of the second detection area. Based on the first detection data and the second detection data, the first reconstructed image covering the first detection area and the second detection area can be reconstructed, i.e., the first reconstructed image can cover an area that cannot be detected by the second detector, and thus the accurate reconstruction range of the reconstructed image can be expanded. (3) The scanning FOV of the first scanning system can be expanded efficiently by adding at least one additional detector module and/or adjusting the arrangement curvature on at least one side of the initial first detector. (4) By processing the first detection data and the second detection data separately, obtaining artifact residuals contained in the reconstructed image corresponding to the extended data for the relatively small scanning FOV, and removing the artifact residuals from the reconstructed image corresponding to the extended data, image artifacts can be removed effectively and accurately, and the reconstructed image for the relatively large scanning FOV can be reconstructed, thereby improving the accuracy of the image reconstruction.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram of a cross-sectional view of an exemplary dual-source scanning system according to some embodiments of the present disclosure;

FIG. 2A is a schematic diagram of a cross-sectional view of an exemplary dual-source scanning system according to some embodiments of the present disclosure;

FIG. 2B is a schematic diagram of an exemplary adjustment of a scanning angle of a first asymmetric detector during axial scanning according to some embodiments of the present disclosure;

FIG. 3A is a schematic diagram of curves representing relationships between a reconstruction FOV corresponding to any location point between a first scanning FOV and an original scanning FOV and angular ranges covered by an X-ray during axial scanning according to some embodiments of the present disclosure;

FIG. 3B is a schematic diagram of exemplary scanning FOVs obtained from two adjacent helical scans during helical scanning according to some embodiments of the present disclosure;

FIG. 4A is a schematic diagram of an exemplary dual-source scanning system according to some other embodiments of the present disclosure;

FIG. 4B is a schematic diagram of an exemplary dual-source scanning system according to some other embodiments of the present disclosure;

FIG. 5 is a flowchart of an exemplary process for controlling a dual-source scanning system according to some embodiments of the present disclosure;

FIG. 6 is a flowchart of an exemplary imaging process according to some embodiments of the present disclosure;

FIG. 7 is a flowchart of an exemplary imaging process according to some other embodiments of the present disclosure;

FIG. 8 is a flowchart of an exemplary imaging process according to some other embodiments of the present disclosure;

FIG. 9 is a schematic diagram illustrating a determination of exemplary virtual detection data according to some embodiments of the present disclosure;

FIG. 10 is a schematic diagram illustrating comparison of errors in a first reconstructed image obtained using an imaging method according to some embodiments of the present disclosure;

FIG. 11 is a schematic diagram of an exemplary imaging device according to some embodiments of the present disclosure;

FIG. 12 is a schematic diagram of an exemplary imaging system according to some embodiments of the present disclosure; and

FIG. 13 is a schematic diagram of an exemplary electronic device according to some embodiments of the present disclosure.

### DETAILED DESCRIPTION

In order to more clearly illustrate the technical solutions of the embodiments of the present disclosure, the accompanying drawings to be used in the description of the embodiments will be briefly described below. Obviously, the accompanying drawings in the following description are only some examples or embodiments of the present disclosure, and that the present disclosure may be applied to other similar scenarios in accordance with these drawings without creative labor for those of ordinary skill in the art. Unless obviously acquired from the context or the context illustrates otherwise, the same numeral in the drawings refers to the same structure or operation.

It should be understood that “system,” “device,” “unit,” and/or “module” as used herein is a way to distinguish between different components, elements, parts, sections, or assemblies at different levels. However, these words may be replaced by other expressions if they accomplish the same purpose.

As indicated in the present disclosure and in the claims, the singular forms “a,” “an,” and “the” may be intended to include the plural forms as well, unless the context clearly indicates otherwise. In general, the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof.

Flowcharts are used in the present disclosure to illustrate the operations performed by the system according to some embodiments of the present disclosure. It should be understood that the operations described herein are not necessarily executed in a specific order. Instead, the operations may be executed in reverse order or simultaneously. Additionally, one or more other operations may be added to these processes, or one or more operations may be removed from these processes.

A dual-source scanning device is a device that simultaneously acquires images of a scanned object using two X-ray bulb systems and two detectors. For example, the dual-source scanning device may reconstruct an image of a detected object located in a detection area based on detection data obtained by using the two detectors separately to scan the scanned object. Since the dual-source scanning device is limited by reasonable geometric design and gantry space, typically the two detectors have different field of views (FOVs), with one detector having a relatively large scanning FOV and the other detector having a relatively small scanning FOV.

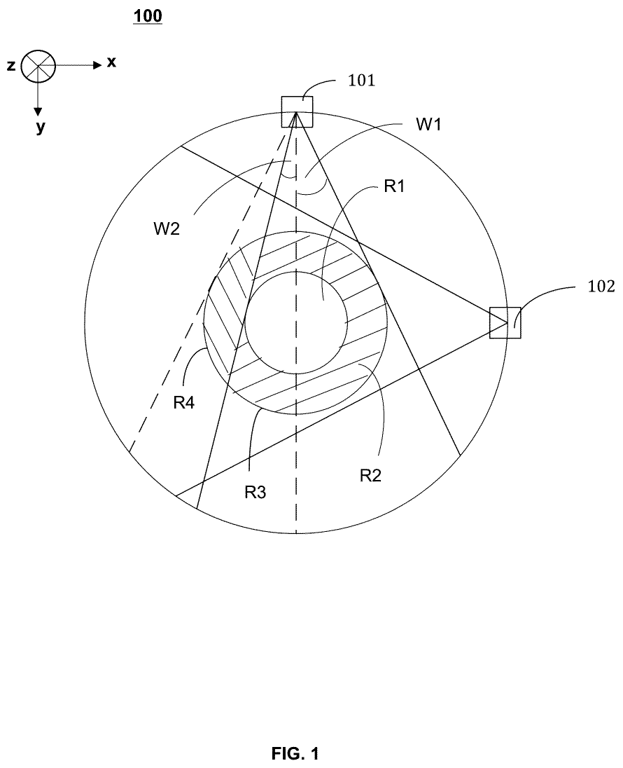

In dual-energy imaging, a reconstruction result is generally displayed only within the scanning field of the detector with the relatively small scanning FOV, or the reconstructed image is marked with an associated marker to prompt a user with an exact image range. Moreover, data acquired by the detector with the relatively large scanning FOV may be not enough to accurately reconstruct images outside the scanning field of the detector with the relatively small scanning FOV. As a result, an image reconstruction range of the dual-source scanning device is limited by the scanning field of the detector with the relatively small scanning FOV. Based on this, some embodiments of the present disclosure provide a dual-source scanning system and a control method thereof, wherein the detector with the relatively small scanning FOV in the dual-source scanning system has an asymmetric structure (e.g., on the basis of a symmetric detector structure, a detector module is added to a side of the detector to expand the scanning field of the side), thereby enhancing the scanning field of the detector with the relatively small scanning FOV. More descriptions of the dual-source scanning system may be found in the relevant descriptions of FIGS. 1-4B.

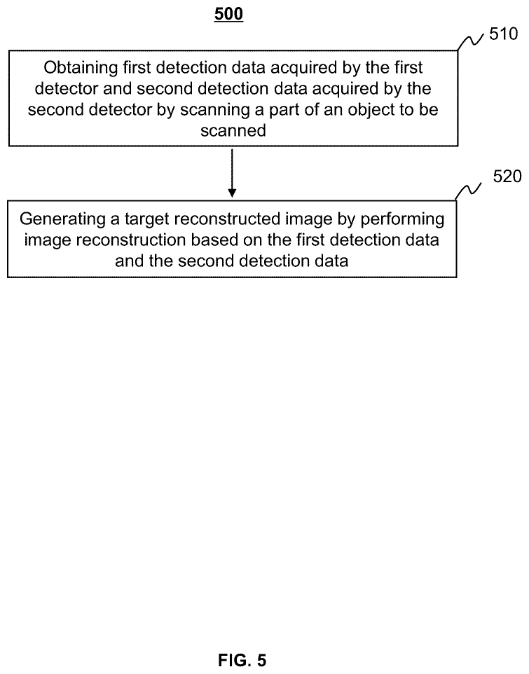

For dual-source scanning devices, if the field of view to be observed includes the entire body of a patient, the detector is then required to have a relatively large scanning field of view, since the detector with the relatively small scanning FOV has a relatively small reconstruction range. For an area outside the scanning field of the detector with the relatively small scanning FOV, if the reconstructed image generated by the detector with the relatively large scanning FOV is mapped to another energy spectrum, then energy spectrum decomposition is performed to obtain a result of the energy spectrum decomposition of the area outside the small detector system. If an image of the area outside the small detector system is essentially from monoenergetic data, the result of the energy spectrum decomposition for the area may be inaccurate. Based on this, some embodiments of the present disclosure provide an imaging method and an imaging system capable of reconstructing scanning data of an asymmetric detector with a relatively small scanning FOV and a detector with a relatively large scanning FOV through a reconstruction method to obtain a reconstructed image within the scanning field of view corresponding to a long side of the asymmetric detector. Compared to dual-source scanning devices using symmetric detector structures, the imaging method of the present disclosure effectively expands the reconstruction range of the images. More descriptions of the imaging method may be found in the related descriptions of FIGS. 5-9.

In some embodiments, a dual-source scanning system may include a first detector and a second detector, and at least one of the first detector or the second detector may have an asymmetric structure relative to a central channel thereof. The first detector has a first scanning field of view (FOV) and the second detector has a second scanning field of view (FOV). The first scanning FOV is a scanning area that the first detector can capture in a single scan (e.g., the scanning area that the beams that are emitted by a first radiation source corresponding to the first detector and received by the first detector can scan). The second scanning FOV is a scanning area that the second detector can capture in a single scan (e.g., the scanning area that the beams that are emitted by a second radiation source corresponding to the second detector and received by the first detector can scan). The first scanning FOV and the second scanning FOV may be at least partially overlapped.

The asymmetric structure relative to a central channel of a detector means that the structure on both sides of the center channel of the detector does not coincide with each other.

In some embodiments, the asymmetric structure is configured as: counts of detector modules on two sides relative to the central channel being different. For example, a count of detector modules on one side relative to the central channel is greater than that of another side relative to the central channel.

In some embodiments, the first detector and the second detector may each include a plurality of detector modules. In some embodiments, the first detector and the second detector may include different numbers of detector modules. In some embodiments, the number of detector modules of the first detector is smaller than the number of detector module of the second detector.

In some embodiments, the asymmetric structure is configured as: arrangement curvatures of detector modules on two sides relative to the central channel being different. For example, an arrangement curvature of detector modules on one side relative to the central channel is greater than that of another side relative to the central channel.

The arrangement curvature refers to a curvature of an overall structure of a plurality of connected detector modules of a detector relative to the corresponding focal point of the detector. For example, the arrangement curvature of the detector modules corresponding to the second detector is the curvature of the second detector relative to a second focal point of the second detector, the second focal point being the focal point of the second detector.

In some embodiments, a scan pitch of the dual-source scanning system is linked to the first scanning FOV of the first detector, wherein: a maximum pitch of the dual-source scanning system may be related to at least one of a distance from the focal point of the first detector to the isocenter, a distance from any point in the first scanning FOV to the isocenter, or a count of detector modules of the first detector in a movement direction of a couch of the dual-source scanning system. More descriptions of this embodiment may be found in operation 620 and its related descriptions.

In some embodiments, the first detector has an asymmetric structure relative to a first central channel of the first detector, the second detector has a symmetric structure relative to a second central channel of the second detector. The first detector with the asymmetric structure relative to the first central channel includes an initial first detector, and field angles on two sides of a central channel of the initial first detector are equal, the first central channel being a central channel of the initial first detector. The field angle is an angle between an outermost ray beam of a plurality of ray beams emitted from the radiation source and received by the detector and a centerline. The centerline is a line that connects the focal point of the detector with an isocenter of the dual-source scanning system, and the centerline is perpendicular to the central channel of the detector.

FIG. 1 is a schematic diagram of a cross-sectional view of an exemplary dual-source scanning system according to some embodiments of the present disclosure. The Z-direction is the same as a movement direction of a couch of the dual-source scanning system.

As shown in FIG. 1, in the dual-source scanning system 100, 101 denotes an asymmetric first detector, 102 denotes a symmetric second detector, W1 denotes a field angle on one side of the first central channel of the first detector 101, W2 denotes a field angle on another side of the first central channel of the first detector 101, and W1 and W2 are unequal. R1 denotes a first detection area, R2 denotes a second detection area, the first detection area R1 is a circular area, the second detection area R2 is an annular area outside the circular area, and the first detection area R1 and the second detection area R2 do not intersect. The second detection area R2 includes an area R3 and an area R4, the area R3 being an area that can be covered by the scanning FOV of the first detector 101, and the area R4 being an area that cannot be covered by the scanning FOV of the first detector 101, i.e., an area to be expanded. In some embodiments, there may be no R4, and R3 may be a whole annular area.

For example, when the first detector 101 and the second detector 102 scan the same object, the second scanning FOV of the second detector 102 includes the first detection area R1 and the second detection area R2, and the first scanning FOV of the first detector 101 includes the first detection area R1 and at least a portion of the second detection area R2 (e.g., detection area R3).

In some embodiments, the first detector with the asymmetric structure relative to the first central channel may include at least one detector module, the at least one detector module having an asymmetric structure with respect to the first central channel.

In some embodiments, the asymmetric structure of the first detector may be configured as counts of detector modules on two sides of the first central channel being unequal, and/or arrangement curvatures of the detector modules on the two sides of the first central channel being different.

In some embodiments, the symmetrical second detector may include at least two detector modules. The at least two detector modules may be symmetrically structured with respect to the second central channel of the second detector.

In some embodiments, the symmetrical structure of the second detector may be configured as: counts of detector modules on two sides of the second central channel being equal and/or arrangement curvatures of the detector modules on the two sides of the second central channel being the same.

Directions of extension of the first central channel of the first detector and the second central channel of the second detector are parallel to a movement direction of a couch of the dual-source scanning system.

In some embodiments, the asymmetric structure of the first detector may be configured as: a count of detector modules on a side close to the second focal point relative to the first central channel of the first detector being greater than a count of detector modules on a side away from the second focal point relative to the first central channel of the first detector, the second focal point being the focal point of the second detector.

In some embodiments, the asymmetric structure of the first detector may be configured as: the arrangement curvature of detector modules on a side close to the second focal point relative to the first central channel of the first detector being different from the arrangement curvature of detector modules on a side away from the second focal point relative to the first central channel of the first detector.

In some embodiments, the asymmetric structure of the first detector may be configured as: the count of detector modules on a side close to the second focal point relative to the first central channel of the first detector being greater than the count of detector modules on a side away from the second focal point relative to the first central channel of the first detector, and the arrangement curvature of detector modules on a side close to the second focal point relative to the first central channel of the first detector being different from the arrangement curvature of detector modules on a side away from the second focal point relative to the first central channel of the first detector.

In some embodiments, the first detector with the asymmetric structure relative to the first central channel may be obtained by: adding at least one additional detector module to at least one side of a symmetric initial first detector.

The initial first detector is a symmetrical portion of the first detector with the asymmetric structure relative to the first central channel. Field angles on two sides of the central channel of the initial first detector are equal. A scanning FOV (hereinafter referred to as original scanning FOV) of the initial first detector is symmetrical relative to the central channel of the initial first detector. The original scanning FOV is smaller than the second scanning FOV.

In some embodiments, the scanning FOV of the at least one additional detector module is an extended scanning FOV. In some embodiments, the extended scanning FOV is located on a side of the original scanning FOV relative to the first central channel of the first detector. In some embodiments, the extended scanning FOV is located on two sides of the original scanning FOV relative to the first central channel of the first detector, wherein a part of the extended scanning FOV located on a side close to the second focal point is greater than another part of the extended scanning FOV on a side away from the second focal point. The first scanning FOV is a sum of the extended scanning FOV and the original scanning FOV.

In some embodiments, the at least one additional detector module may be added in a variety of ways. For example, at least one additional detector module may be added to a side of the initial first detector close to the second focal point and a side away from the second focal point, respectively, and a count of additional detector modules added to the side close to the second focal point may be greater than a count of additional detector modules added to the side away from the second focal point. As another example, at least one additional detector module may be added to the side of the initial first detector close to the second focal point, while keeping the count of initial detector modules on the side away from the second focal point unchanged.

In some embodiments, the count of additional detector modules may be related to a gap between hardware devices, and a maximum count of additional detector modules is required to ensure that the dual-source scanning system operates without interference between the hardware devices. For example, when adding additional detector modules on the side close to the second focal point, it is necessary to ensure that there is no interference between the added detector modules and a ray generator of the second detector. For example, when adding the additional detector modules on the side away from the second focal point, it is necessary to ensure that there is no interference between the added detector modules and the second detector.

In some embodiments, the first detector may be obtained by: adjusting an arrangement curvature on at least one side of the symmetric initial first detector.

In some embodiments, the arrangement curvature on the at least one side of the initial first detector may be adjusted in a variety of ways. For example, the arrangement curvature on the side of the initial first detector close to the second focal point and the arrangement curvature on the side away from the second focal point may be adjusted (e.g., increased or decreased) separately such that the arrangement curvature on the side close to the second focal point is smaller than the arrangement curvature on the side away from the second focal point. As another example, the arrangement curvature on the side of the initial first detector near the second focal point may be reduced and the arrangement curvature on the side of the initial first detector away from the second focal point may be kept constant. As yet another example, the arrangement curvature on the side of the initial first detector near the second focal point may be kept constant, and the arrangement curvature on the side of the initial first detector away from the second focal point may be increased.

In some embodiments, the first detector may be obtained by: adding at least one additional detector module to at least one side of a symmetric initial first detector, and adjusting an arrangement curvature on at least one side of the symmetric initial first detector.

In some embodiments, the symmetrical initial first detector may include at least two initial detector modules, and the arrangement curvature of the at least one additional detector module may be smaller than an arrangement curvature of the at least two initial detector modules. For example, when adding the at least one additional detector module to the side of the initial first detector near the second focal point, the arrangement curvature of each of the at least one additional detector module may be set to be smaller than the arrangement curvature of the at least two initial detector modules.

The at least two initial detector modules may have a symmetrical structure with respect to the first central channel. More descriptions of the symmetrical structure may be found in the above related descriptions.

In some embodiments, the arrangement curvature of a portion of the at least one additional detector module may be smaller than an arrangement curvature of the at least two initial detector modules. For example, when adding the at least one added detector module to the side of the initial first detector close to the second focal point, the at least one added detector module may be configured such that the arrangement curvature of a portion of the at least one additional detector module is smaller than the arrangement curvature of the at least two initial detector modules.

In some embodiments of the present disclosure, by configuring the first detector as an asymmetric structure, the scanning FOV of the asymmetric first detector can be effectively enlarged to improve the reconstruction accuracy and expand the reconstruction range. The scanning FOV of the asymmetric first detector can be effectively enlarged by adding at least one additional detector module, and/or by adjusting the arrangement curvature on at least one side of the initial first detector.

In some embodiments, the first scanning FOV of the first detector with the asymmetric structure relative to the first central channel includes a first detection area and at least a part of a second detection area, and the second scanning FOV of the second detector with the symmetric structure relative to the second central channel includes the first detection area and the second detection area, wherein the first detection area is surrounded by the second detection area, and the first scanning FOV of the first detector is smaller than the second scanning FOV of the second detector.

In some embodiments, the first scanning FOV of the first detector includes the first detection area and and the second detection area, the second scanning FOV of the second detector includes the first detection area, the second detection area and an additional detection area. The additional detection area may be a portion of the detection area in the second scanning FOV of the second detector that is not intersected by the second detection area. That is, the detection area under the second scanning FOV of the second detector is a sum of the first detection area, the second detection area, and the additional detection area.

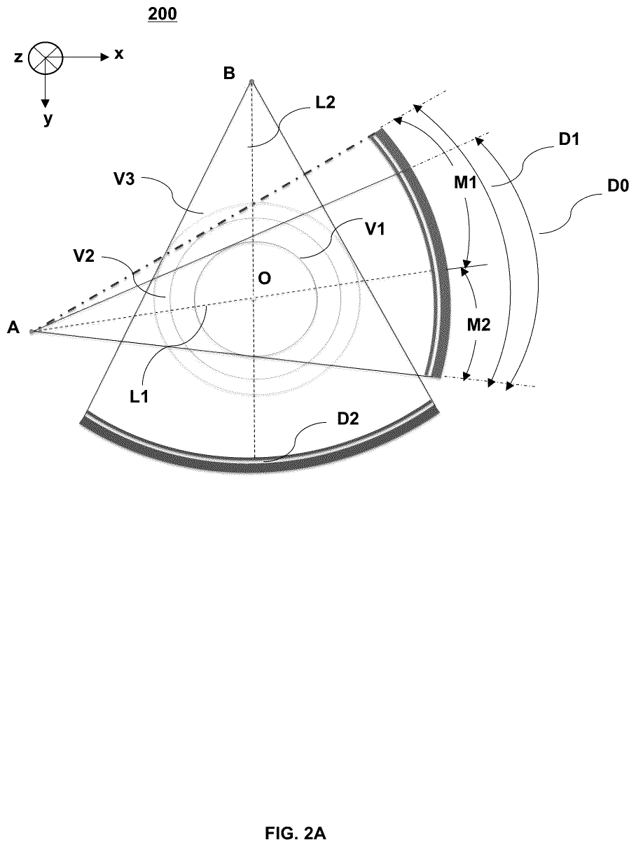

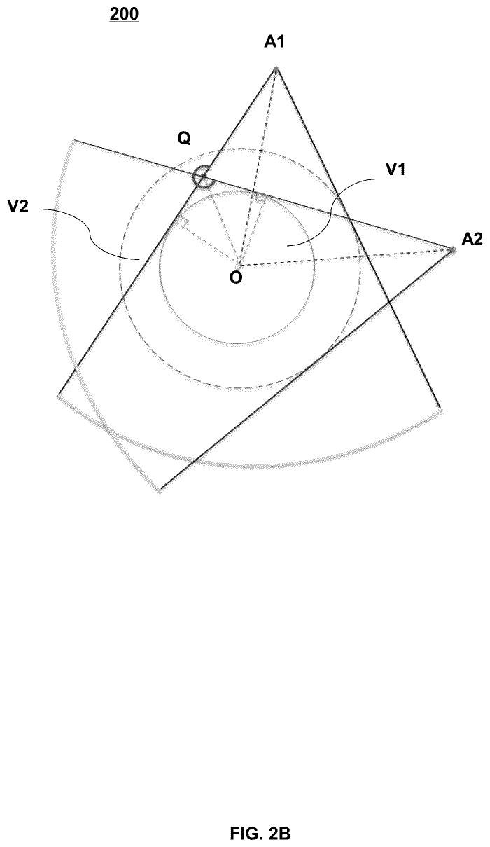

FIG. 2A is a schematic diagram of a cross-sectional view of an exemplary dual-source scanning system in the Z-direction according to some embodiments of the present disclosure. Some of the following embodiments may be understood with reference to FIG. 2A, but the accompanying drawings are only for illustration of some of these embodiments and do not constitute a limitation of the embodiments.

As shown in FIG. 2A, in the dual-source scanning system 200, A denotes a first focal point of an asymmetric first detector, DO denotes a symmetric initial first detector, D1 denotes an asymmetric first detector, and L1 denotes a first centerline. B denotes a second focal point of a symmetric second detector, D2 denotes a symmetric second detector, and L2 denotes a second centerline. V1 denotes a first detection area, and V2 denotes a second detection area, V3 denotes an additional detection area, O denotes a isocenter, M1 denotes a side of the asymmetric first detector DO close to the second focal point B, and M2 denotes a side of the asymmetric first detector D1 away from the second focal point B. The first centerline L1 is a centerline of the asymmetric first detector D1, and the first centerline L1 connects the first focal point A with the isocenter O and is perpendicular to the first central channel of the asymmetric first detector D1. The second centerline L2 is a centerline of the symmetric second detector D2, and the second centerline L2 connects the second focal point B with the isocenter O and is perpendicular to the second central channel of the symmetric second detector D2. The first detection area V1 is a circular area, the second detection area V2 is a ring area outside the first detection area V1, and the additional detection area V3 is a ring area outside the second detection area V2. The first detection area V1 does not intersect with the second detection area V2, and the second detection area V2 does not intersect with the additional detection area V3. The first focal point A is the focal point of the first detector. The second focal point B is the focal point of the second detector. The isocenter O is a center point of the dual-source detector when the dual-source scanning system performs rotational scanning.

As shown in FIG. 2A, the symmetric second detector D2 is a symmetrical structure with respect to the second centerline L2. A count of detector modules on the side M1 close to the second focal point B may be greater than a count of detector modules on the side M2 away from the second focal point B, such that the asymmetric first detector D1 has an asymmetric structure with respect to the first centerline L1.

In some embodiments, when the asymmetric first detector and the symmetric second detector scan a same object, the first scanning FOV of the asymmetric first detector D1 includes the first detection area V1 and a portion of the second detection area V2, and the second scanning FOV of the symmetric second detector D2 includes the first detection area V1 and the second detection area V2.

In some embodiments, when the asymmetric first detector and the symmetric second detector scan the same object, the first scanning FOV of the asymmetric first detector D1 includes the first detection area V1 and the second detection area V2, and the second scanning FOV of the symmetric second detector D2 includes the first detection area V1, the second detection area V2, and the additional detection area V3.

In some embodiments, when the asymmetric first detector and the symmetric second detector scan a same object, the first scanning FOV of the asymmetric first detector D1 includes the first detection area V1, the second detection area V2, and a portion of the additional detection area V3, and the second scanning FOV of the symmetric second detector D2 includes the first detection area V1, the second detection area V2, and the additional detection area V3.

It should be noted that the first detection area V1 described above may be a detection area under the original scanning FOV of the initial first detector. The second detection area V2 may be a detection area under the extended scanning FOV of the first detector. The additional detection V3 area may be a portion of the detection area under the second scanning FOV of the second detector that is not intersected by the second detection area.

In some embodiments, when performing axial scanning using the dual-source scanning system described in some embodiments of the present disclosure, the scanning angle associated with the first focal point of the first scanning system may be adjusted around the isocenter. In some embodiments, an angle between the asymmetric first scanning system and the symmetric second scanning system may be 90° or any other feasible angle, and the asymmetric first scanning system and the symmetric second scanning system may maintain a constant relative position as the dual-source scanning device rotates.

FIG. 2B is a schematic diagram of an exemplary adjustment of a scanning angle of a first asymmetric detector during axial scanning according to some embodiments of the present disclosure. As shown in FIG. 2B, A1 denotes a scanning position of a first focal point, A2 denotes another scanning position of the first focal point, and O denotes a isocenter; an original scanning FOV corresponding to an initial first detector of the first detector includes a first detection area V1, and a first scanning FOV corresponding to an asymmetric first detector includes a sum of the first detection area V1 and a second detection area V2. Point Q denotes a location point between the first detection area V1 and the second detection area V2.

In some embodiments, the first detector has an asymmetric structure relative to a first central channel of the first detector, the second detector has an asymmetric structure relative to a second central channel of the second detector.

In some embodiments, the second detector with the asymmetric structure relative to the second central channel may include at least one detector module, the at least one detector module having an asymmetric structure with respect to the second central channel.

In some embodiments, the asymmetric structure of the second detector may be configured as counts of detector modules on two sides of the second central channel being unequal, and/or arrangement curvatures of the detector modules on the two sides of the second central channel being different.

The second detector with the asymmetric structure is similar to the first detector with the asymmetric structure, the difference being that the second detector with the asymmetric structure has a larger scanning FOV than the first detector with the asymmetric structure. More descriptions of the asymmetrical structure may be found in the above related descriptions.

In some embodiments, wherein the asymmetric structure of the second detector is configured as: a count of detector modules on a side close to a first focal point relative to the second central channel of the second detector being greater than a count of detector modules on a side away from the first focal point relative to the second central channel of the second detector, the first focal point being the focal point of the first detector.

In some embodiments, the asymmetric structure of the second detector may be configured as: the arrangement curvature of detector modules on a side close to the first focal point relative to the second central channel of the second detector being different from the arrangement curvature of detector modules on a side away from the first focal point relative to the second central channel of the second detector.

In some embodiments, the asymmetric structure of the second detector may be configured as: the count of detector modules on a side close to the first focal point relative to the second central channel of the second detector being greater than the count of detector modules on a side away from the first focal point relative to the second central channel of the second detector, and the arrangement curvature of detector modules on a side close to the first focal point relative to the second central channel of the second detector being different from the arrangement curvature of detector modules on a side away from the first focal point relative to the second central channel of the second detector.

In some embodiments, the second detector with the asymmetric structure relative to the second central channel may be obtained by the same way in which the first detector with the asymmetric structure relative to the first central channel is obtained. More descriptions of the way in which the asymmetrical structure is obtained may be found in the above related descriptions.

In some embodiments, the first scanning FOV of the first detector includes at least a part of a first detection area and at least a part of a second detection area, and the second scanning FOV of the second detector includes at least a part of the first detection area and at least a part of the second detection area, the first detection area being surrounded by the second detection area; or the first scanning FOV of the first detector includes at least a part of a third detection area, and the second scanning FOV of the second detector includes at least a part of a fourth detection area, the third detection area and the fourth detection area are partially overlapped.

In some embodiments, the first scanning FOV of the first detector includes the first detection area and a part of the second detection area, the second scanning FOV of the second detector includes the first detection area and a part of the second detection area. In some embodiments, the first scanning FOV of the first detector includes a part of the first detection area and a part of the second detection area, the second scanning FOV of the second detector includes a part of the first detection area and a part of the second detection area. In some embodiments, the first scanning FOV of the first detector includes the first detection area and and a part of the second detection area, the second scanning FOV of the second detector includes the first detection area and the second detection area.

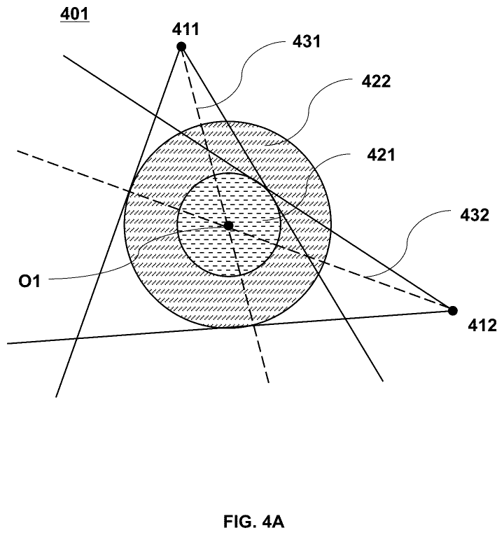

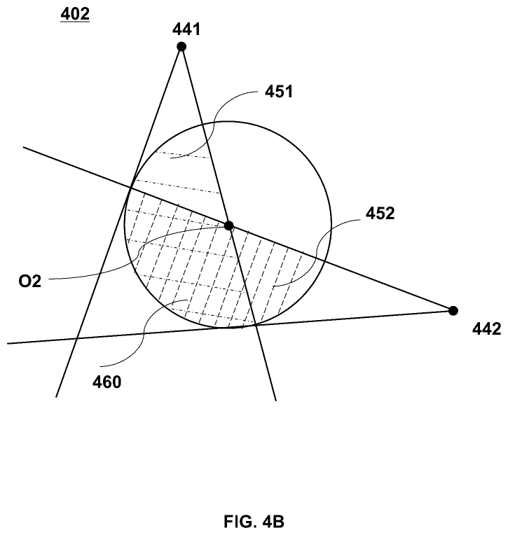

As shown in FIG. 4A, in the dual-source scanning system 401, 411 denotes a first focal point of an asymmetric first detector, 412 denotes a second focal point of an asymmetric second detector, 421 denotes a first detection area, 422 denotes a second detection area, 431 denotes a first centerline, 432 denotes a second centerline, and O1 denotes an isocenter of the dual-source scanning system 401. In some embodiments, the first scanning FOV of the first detector 411 includes at least a part of the first detection area 421 and at least a part of the second detection area 422, and the second scanning FOV of the second detector includes at least a part of the first detection area 421 and at least a part of the second detection area 422, the first detection area 421 being surrounded by the second detection area 422.

In some embodiments, the first scanning FOV of the first detector includes at least a part of a third detection area, the second scanning FOV of the second detector includes at least a part of a fourth detection area, the third detection area and the fourth detection area are partially overlapped.

As shown in FIG. 4B, in the dual-source scanning system 402, 441 denotes a first focal point of an asymmetric first detector, 442 denotes a second focal point of an asymmetric second detector, 451 denotes a third detection area, 452 denotes a fourth detection area, and 02 denotes an isocenter of the dual-source scanning system 402. The third detection area 451 and the fourth detection area 452 are partially overlapped, and 460 denotes an overlap area between the third detection area 451 and the fourth detection area 452. In some embodiments, the first scanning FOV of the first detector 441 includes at least a part of the third detection area 451, and the second scanning FOV of the second detector includes at least a part of the fourth detection area 452.

Some embodiments of the present disclosure also provide a method for controlling a dual-source scanning system that can realize dual-source scanning based on the dual-source scanning system described in any of the above embodiments. More descriptions of the method of controlling the dual-source scanning system may be found in FIGS. 5-9 and its related descriptions.

FIG. 5 is a flowchart of an exemplary process for controlling a dual-source scanning system according to some embodiments of the present disclosure. In some embodiments, the dual-source scanning system may include a processing device, and process 500 may be performed by the processing device. The processing device may establish a data connection with other components (e.g., a first detector, a second detector, etc.) of the dual-source scanning system in a wired manner or a wireless manner, thereby controlling the dual-source scanning system. As shown in FIG. 5, process 500 may include one or more of the following operations.

In some embodiments, the dual-source scanning system includes a first detector and a second detector, at least one of the first detector and the second detector has an asymmetric structure relative to a central channel thereof, the first detector has a first scanning field of view (FOV), the second detector has a second scanning FOV, and the first scanning FOV and the second scanning FOV are at least partially overlapped. More descriptions of dual-source scanning system may be found in the above related descriptions.

In 510, first detection data acquired by the first detector and second detection data acquired by the second detector by scanning a part of an object to be scanned may be obtained.

In some embodiments, the object to be scanned may be biological or non-biological. For example, the object to be scanned may include a patient, a man-made object, or the like.

In some embodiments, the part to be scanned may include a particular portion of the body, e.g., the head, neck, chest, etc., or any combination thereof. In some embodiments, the part to be scanned may include a specific organ, e.g., the liver, kidney, pancreas, bladder, uterus, rectum, etc., or any combination thereof. In some embodiments, the part to be scanned may include an area of interest (ROI), e.g., a tumor, a nodule, or the like. The relevant descriptions of the object to be scanned and the part to be scanned are for illustrative purposes only and are not intended to limit the scope of the present disclosure.

The processing device may determine the part of the object to be scanned in a variety of ways. For example, the processing device may determine the part to be scanned based on a user input. As another example, the processing device may determine the part to be scanned based on a scanning protocol. The embodiments of the present disclosure do not have any special limitation on the manner of determining the part to be scanned, and a person of ordinary skill in the art may perform a familiar operation to determine the part to be scanned.

In some embodiments, the first detector has a first scanning FOV, the second detector has a second scanning FOV, and the first scanning FOV of the first detector and the second scanning FOV of the second detector are at least partially overlapped. More descriptions of the first detector and the second detector may be found in the preceding related descriptions.

The first detection data refers to relevant data obtained by scanning the part to be scanned using the first detector. The second detection data refers to relevant data obtained by scanning the part to be scanned using the second detector. The first detection data and the second detection data correspond to a same part to be scanned.

In some embodiments, the first detection data and the second detection data may be raw scanning data. For example, the first detection data and the second detection data may include a signal value for each position of the part to be scanned. A signal value indicates a signal strength in a logarithmic domain.

In some embodiments, each of the first detector and the second detector has an energy spectrum exposure parameter. The energy spectrum exposure parameter refers to an energy parameter of the X-rays emitted by the detector. The first detection data is detection data collected when the first detector scans the part to be scanned using a first energy spectrum, and the second detection data is detection data collected when the second detector scans the part to be scanned using a second energy spectrum. The energy spectrum exposure parameters of the first detector and the second detector may be the same, in this case, the first energy spectrum and the second energy spectrum are equal.

In some embodiments, the energy spectrum exposure parameter of the first detector and the energy spectrum exposure parameter of the second detector may be different, and the first energy spectrum is higher than the second energy spectrum. By scanning a same part to be scanned with two detectors having different energy spectrum exposure parameters in order to obtain the first detection data and the second detection data, the accuracy of the reconstructed image of the part to be scanned obtained subsequently can be improved.

The first detection data and the second detection data may be obtained in a variety of ways. By way of example, the processing device may obtain the first detection data and the second detection data from other components (e.g., a first detector, a second detector, etc.) of the dual-source scanning system, respectively, or the processing device may obtain the first detection data from the first detector and the second detection data from the second detector from a server, in which scenario the first detection data and the second detection data is stored in the server.

As another example, an electronic device may trigger other components (e.g., the asymmetric first detector, the symmetric second detector, etc.) of the dual-source scanning system to scan the part to be scanned to cause the first detector to scan the part to be scanned using the first energy spectrum to obtain the first detection data, and to cause the second detector to scan the part to be scanned using the second energy spectrum to obtain the second detection data. The triggering manner may include sending control commands to the first detector and the second detector. Alternatively, the other components (e.g., the first detector, the second detector, etc.) of the dual-source scanning system may be triggered by the user to scan the part to be scanned, and the triggering manner is not limited by the present disclosure.

In 520, a target reconstructed image may be generated by performing image reconstruction based on the first detection data and the second detection data.

The target reconstructed image refers to a reconstructed image obtained after removing artifacts. Image reconstruction refers to an operation of converting detection data into a 2D or 3D image. The detection data may be obtained when a detector performs scanning at a plurality of angles. In some embodiments, the target reconstructed image is the final reconstructed image of the dual-source scanning system.

In some embodiments, the target reconstructed image is a reconstructed image corresponding to the second scanning FOV. In other words, the target reconstructed image may be understood as a reconstructed image obtained after expanding the first scanning FOV to be equal to the second scanning FOV by performing the extension operation on the first detection data.

The target reconstructed image may be determined in various ways. In some embodiments, the processing device may reconstruct image(s) based on the second detection data and the first detection data using various reconstruction techniques. For example, the processing device may perform reconstruction using a filtered inverse projection algorithm, an iterative reconstruction algorithm, an analytical technique, or the like, and the embodiments of the present disclosure do not limit the specific algorithm used for image reconstruction.

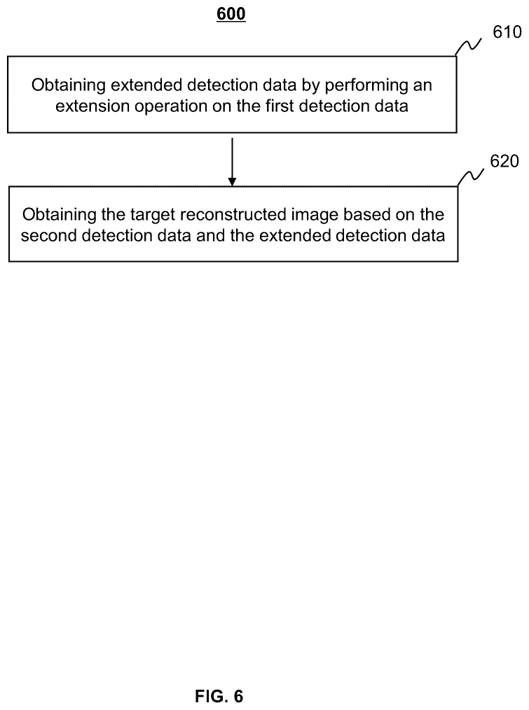

In some embodiments, if the first scanning FOV includes the first detection area and a part of the second detection area, the second scanning FOV includes the first detection area and the second detection area, and the first detection area being surrounded by the second detection area, the processing device may obtain extended detection data by performing an extension operation on the first detection data, and obtain the target reconstructed image based on the second detection data and the extended detection data. More descriptions of this embodiment may be found in FIG. 6 and its related descriptions.

In some embodiments, when performing scanning using the dual-source scanning system described in some embodiments of the present disclosure, for any location point in the first scanning FOV, a reconstruction FOV corresponding to the location point correlates to an angular range in which a first focal point irradiates the location point. In some embodiments, when performing axial scanning using the dual-source scanning system described in some embodiments of the present disclosure, for any location point in the first scanning FOV, a reconstruction FOV corresponding to the location point correlates to an angular range in which the first focal point irradiates the location point. In some embodiments, when performing helical scanning using the dual-source scanning system described in some embodiments of the present disclosure, for any location point in the first scanning FOV, a reconstruction FOV corresponding to the location point correlates to an angular range in which the first focal point irradiates the location point.

It should be noted that during the operation of the dual-source scanning system, the first focal point may irradiate any location point between the first scanning FOV and the original scanning FOV over a section of consecutive angles of rotation. The section of angles of rotation and the isocenter form a consecutive angular range, i.e., the angular range in which the first focal point irradiates the location point.

In some embodiments, the angular range of the first focal point irradiating the location point may be obtained by Equation (1):

$$α = π + 2 \times sin ( \frac{0.5 \times {D}_{FOV 0}}{r} ) , \qquad ( 1 )$$

wherein α denotes the angular range of the first focal point irradiating a location point, D_{FOV0} denotes a diameter of the original scanning FOV, and r denotes a distance from the location point to the isocenter.

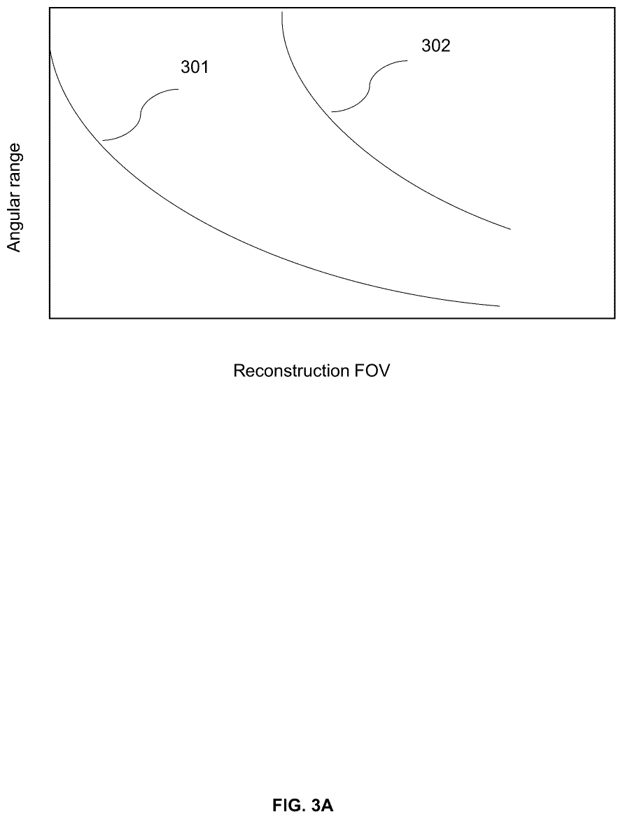

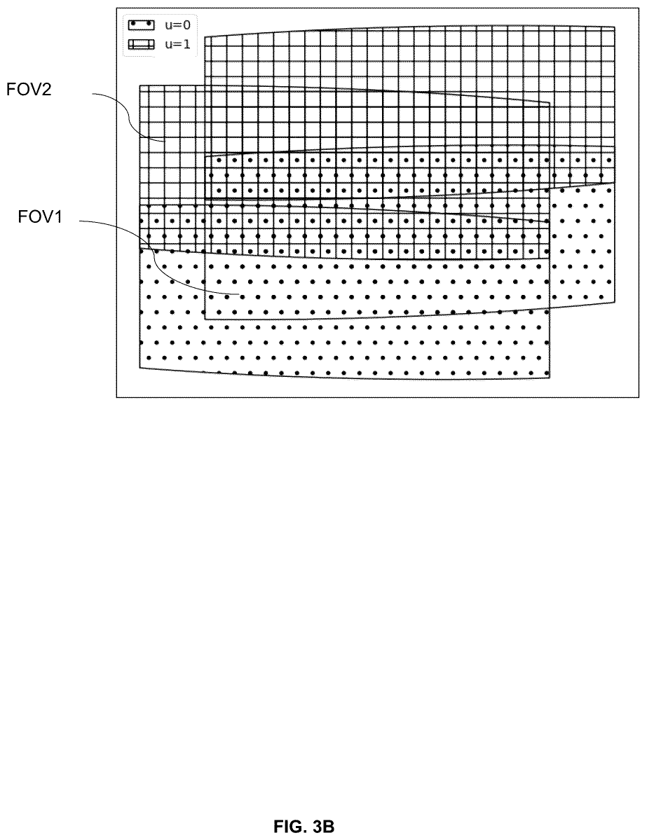

In some embodiments, the reconstruction FOV corresponding to any location point between the first scanning FOV and the original scanning FOV may be negatively correlated to the angular range of the first focal point irradiating the location point. FIG. 3A is a schematic diagram of curves representing relationships between a reconstruction FOV corresponding to any location point between a first scanning FOV and an original scanning FOV and angular ranges covered by an X-ray during axial scanning according to some embodiments of the present disclosure. Curve 301 is a curve representing a relationship between the reconstruction FOV corresponding to any location point between the second scanning FOV and the original scanning FOV and an angular range in which beams from a second focal point irradiates the location point. Curve 302 is a curve representing a relationship between the reconstruction FOV corresponding to any location point between the first scanning FOV and the original scanning FOV and an angular range in which beams from a first focal point irradiates the location point. The horizontal coordinates of the curves represent the reconstruction FOV corresponding to any location point between the first scanning FOV and the original scanning FOV, and the vertical coordinate represent the angular range covered by the X-ray (e.g., the angular range in which the first focal point and the second focal point irradiates the location point, respectively). As can be seen from FIG. 3A, the reconstruction FOV corresponding to any location point between the first scanning FOV and the original scanning FOV is negatively correlated with the angular range of the first focal point irradiating location point, and the reconstruction FOV corresponding to any location point between the second scanning FOV and the original scanning FOV is negatively correlated with the angular range of the second focal point irradiating location point.

In some embodiments, when performing scanning using the dual-source scanning system described in some embodiments of the present disclosure, a width difference between a width of a reconstruction FOV of a previous scan in a movement direction of the couch and a width of a reconstruction FOV of a subsequent scan in the movement direction of the couch satisfies a preset condition. In some embodiments, when performing helical scanning using the dual-source scanning system described in some embodiments of the present disclosure, a width difference between a width of a reconstruction FOV of a previous helical scan in the movement direction of the couch and a width of a reconstruction FOV of a subsequent helical scan in the movement direction of the couch satisfies a preset condition. In some embodiments, the preset condition may be that the width difference between the width of the reconstruction FOV of the previous helical scan in the movement direction of the couch and the width of the reconstruction FOV of the subsequent helical scan in the movement direction of the couch is less than a preset threshold. In some embodiments, the preset threshold is related to a width of a single row of detector modules in the movement direction of the couch. For example, the preset threshold may be the width of the single row of detector modules in the movement direction of the couch.

Exemplarily, the preset threshold and the width difference between the width of the reconstruction FOV of the previous helical scan in the movement direction of the couch and the width of the reconstruction FOV of the subsequent helical scan in the movement direction of the couch may satisfy a relationship represented by Equation (2):
wherein:

$${z}_{POV 1} - {z}_{POV 2} \leq S , \qquad ( 2 )$$

$${Z}_{POV 1} = - \frac{{N}_{q} - 1}{2} \times S \times \sqrt{1 - {( b / SID )}^{2}} + {z}_{rot} \times \frac{arc sin ( b / SID )}{2 π} + {Z}_{rot} , and$$

$${Z}_{POV 2} = \frac{{N}_{q} - 1}{2} \times S \times \sqrt{1 - {( b / SID )}^{2}} + {z}_{rot} \times \frac{arc sin ( b / SID )}{2 π} .$$

wherein z_{POV1} denotes a width of a reconstruction FOV of a previous scan in a movement direction of the couch, z_{POV2} denotes a width of a reconstruction FOV of a subsequent scan in the movement direction of the couch, S denotes the width of the single raw of detector modules in the movement direction of the couch, N_{q} denotes a count of layers of detector modules in the first detector in the movement direction of the couch, b denotes a distance from any point in the first scanning FOV to the isocenter, zᵣₒₜ denotes a distance traveled by a medical bed during one rotation of helical scanning performed by the first detector, SID denotes a distance from the first focal point to the isocenter.

In some embodiments, the distance traveled by the medical bed during one rotation of helical scanning performed by the first detector may be obtained by Equation (3):

$${z}_{rot} = {N}_{q} \times S \times P . \qquad ( 3 )$$

wherein P denotes a pitch of the first detector.

FIG. 3B is a schematic diagram of exemplary scanning FOVs obtained from two adjacent helical scans during helical scanning according to some embodiments of the present disclosure. The dot-filled area in the drawing represents a scanning FOV obtained during a first helical scan (i.e., u=0, u is a count of completed rotations), and the box-line-filled area represents a scanning FOV obtained during a second helical scan (i.e., u=1). FOV1 is a first scanning FOV obtained by the first detector when performing helical scanning, and FOV2 is a second scanning FOV obtained by the second detector when performing helical scanning.

In some embodiments, the scanning a part of an object to be scanned includes: scanning the part of the object to be scanned using a helical scan; wherein a maximum pitch of the dual-source scanning system may be related to at least one of a distance from the focal point of the first detector to the isocenter of the dual-source scanning system, a distance from any point in the first scanning FOV to the isocenter, or a count of layers of detector modules in a movement direction of a couch of the dual-source scanning system. The maximum pitch may be determined based on a threshold condition that the pitch needs to satisfy.

In some embodiments, the threshold condition that the pitch needs to satisfy may be represented by Equation (4):

$$P \leq \frac{1 + ( {N}_{q} - 1 ) \times \sqrt{1 - {( {b}_{max} / SID )}^{2}}}{{N}_{q}} . \qquad ( 4 )$$

Accordingly, in some embodiments, the maximum pitch may be calculated by Equation (5):

$${P}_{max} \leq \frac{1 + ( {N}_{q} - 1 ) \times \sqrt{1 - {( {b}_{max} / SID )}^{2}}}{{N}_{q}} , \qquad ( 5 )$$

wherein bₘₐₓ denotes a maximum distance, and Pₘₐₓ denotes the maximum pitch.

In some embodiments, when bₘₐₓ=b₂ (wherein b₂ is a radius of the first scanning FOV,

$${b}_{2} = \frac{{D}_{FOV 2}}{2} ,$$

D_{FOV2} is a diameter of the first scanning FOV, and P satisfies the above threshold condition, it may be ensured that the extended scanning FOV obtained from helical scanning after using the dual-source scanning system as described in some embodiments of the present disclosure is sufficient and effective.

See FIGS. 1-4B and their associated descriptions for more descriptions of the detector module and the isocenter.

In a possible embodiment of the present disclosure, after obtaining the target reconstructed image, the electronic device may output the target reconstructed image. For example, the electronic device may output the target reconstructed image with the aid of a display of the electronic device, or the electronic device may send the target reconstructed image to other devices that need to use the target reconstructed image, which is not limited by the embodiments of the present disclosure.

See FIG. 1, when the dual-source scanning system 100 performs scanning (e.g., axial scanning or helical scanning, etc.), a projection angle of the dual-source scanning system 100 changes, and positions of the asymmetric first detector D1 and the symmetric second detector D2 on the dual-source scanning system 100 change. Since the symmetric second detector D2 is a symmetrically structured detector, when the position of the symmetric second detector D2 changes, the second scanning FOV of the symmetric second detector D2 still includes the first detection area V1 and the second detection area V2. The asymmetric first detector D1 is a detector with an asymmetric structure, so that the first scanning FOV of the asymmetric first detector D1 changes when the position of the asymmetric first detector D1 changes, and the first scanning FOV of the asymmetric first detector D1 still includes a portion of the second detection area V2. When performing scanning using the dual-source scanning system 100, a part of an object to be scanned is located in an area that includes the first detection area V1 and the second detection area V2.

Since the second scanning FOV of the symmetric second detector D2 includes the first detection area V1 and the second detection area V2, and the first scanning FOV corresponding to the asymmetric first detector includes the first detection area V1 and a portion of the second detection area V2, a second reconstructed image capable of covering the first scanning FOV and the second scanning FOV may be reconstructed based on second detection data of the symmetric second detector D2. The first scanning FOV of the asymmetric first detector D1 does not fully cover the second detection area V2, and therefore a reconstructed image capable of covering the second scanning FOV may not be accurately reconstructed only based on first detection data of the asymmetric first detector D1. Some embodiments of the present disclosure also provide an imaging method, an imaging device, and an electronic device capable of accurately reconstructing a first reconstructed image covering the first detection area V1 and the second detection area V2 based on the first detection data and the second detection data, thereby expanding an accurate reconstruction range of the reconstructed image, and obtaining a target reconstructed image with a high degree of accuracy based on the first reconstructed image and the second reconstructed image. More descriptions of the imaging method, the imaging device, and the electronic device may be found in the related descriptions of FIG. 6.

FIG. 6 is a flowchart of an exemplary imaging process according to some embodiments of the present disclosure. In some embodiments, process 600 may be performed by an electronic device. The electronic device may establish a data connection to the dual-source scanning system described in any embodiment of the present disclosure via a wired or wireless manner, to enable control of an imaging device. As shown in FIG. 6, process 600 may include one or more of the following operations.

In 610, extended detection data may be obtained by performing an extension operation on the first detection data.

The extended detection data refers to relevant data obtained after the data extension operation. In some embodiments, the extended detection data may be raw scanning data after the data extension operation.

In some embodiments, a scanning FOV corresponding to the extended detection data may be larger than the first scanning FOV. In some embodiments, the scanning FOV corresponding to the extended detection data may be between the first scanning FOV and the second scanning FOV. In some embodiments, the scanning FOV corresponding to the extended detection data may be equal to the second scanning FOV.

The extension operation refers to an operation of expanding data based on existing data. In some embodiments, the processing device may perform the extension operation by various techniques such as nearest neighbor interpolation, linear interpolation, etc., and the embodiments of the present disclosure do not limit the specific technique used for the extension operation.

In dual-source scanning, due to the different scanning FOVs corresponding to different radioactive sources, a data volume of detection data obtained in a relatively small scanning FOV is smaller than a data volume of detection data obtained in a relatively large scanning FOV. Truncation artifacts are generated when reconstructing a reconstructed image (e.g., a CT image, etc.) corresponding to the relatively large scanning FOV based on the detection data corresponding to the relatively small scanning FOV. Truncation artifacts are usually formed due to insufficient data acquisition.

After obtaining the first detection data, by performing the extension operation on the first detection data, truncation artifacts in data reconstruction for detection data corresponding to the scanning FOV caused by data loss can be reduced to some extent.

In some embodiments, the second scanning FOV of the second detector includes a first detection area (e.g., the first detection area V1) and a second detection area (e.g., the second detection area V2), and the second detection area includes a detection area corresponding to an extended scanning FOV of the first detector and an area to be expanded. The area to be extended is an area within the second detection area but outside the first scanning FOV of the first detector. For example, when the detection area corresponding to the extended scanning FOV is a portion of the second detection area, the area to be expanded is another portion of the second detection area. Merely by way of example, since the first detector has an asymmetric structure, the detection area corresponding to the first scanning FOV is asymmetric, i.e., the area to be expanded may be a mirrored area of the extended scanning FOV with respect to the first centerline.

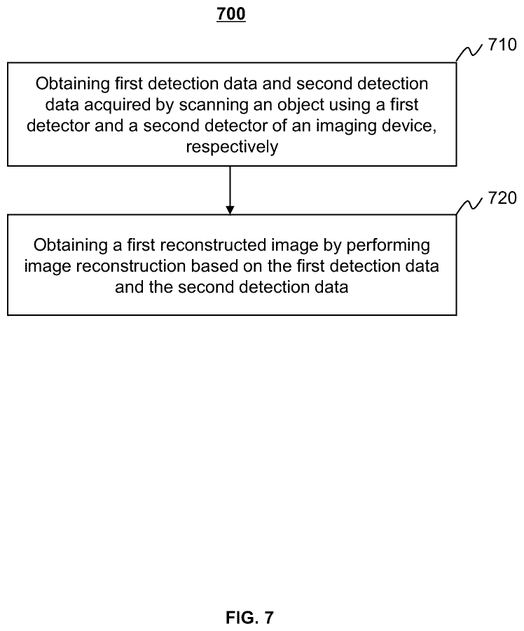

In some embodiments, the processing device may determine virtual detection data for the area to be expanded based on the first detection data and the second detection data, thereby expanding the first detection data of the first detector to obtain the extended detection data, the extended detection data being detection data covering the first detection area and the second detection area. Further, the processing device may reconstruct a first reconstructed image with a high degree of accuracy based on the detection data (i.e., the extended detection data) corresponding to an area covering the first detection area and the second detection area. In this embodiment, the extended detection data is detection data corresponding to an area covering the first detection area and the second detection area. More descriptions of obtaining the virtual detection data may be found in FIG. 7 and its related descriptions. More descriptions of the extended scanning FOV may be found in FIG. 1 and its related descriptions.

By performing the extension operation on the first detection data corresponding to the first scanning FOV, the data volume of the first detection data can be expanded to be greater than the data volume of the first detection data corresponding to the first scanning FOV, thereby reconstructing a reconstructed image with a range larger than the first scanning FOV.

In some embodiments, the scanning FOV of the second detector includes the first detection area, the second detection area, and an additional detection area disposed peripheral to the second detection area. The second detection area includes the detection area corresponding to the extended scanning FOV of the first detector and the area to be expanded, and the additional detection area is a detection area that cannot be detected by the extended scanning FOV of the first detector. The composite data of the first detection data and the virtual detection data corresponds to an area capable of covering the first detection area and the second detection area.

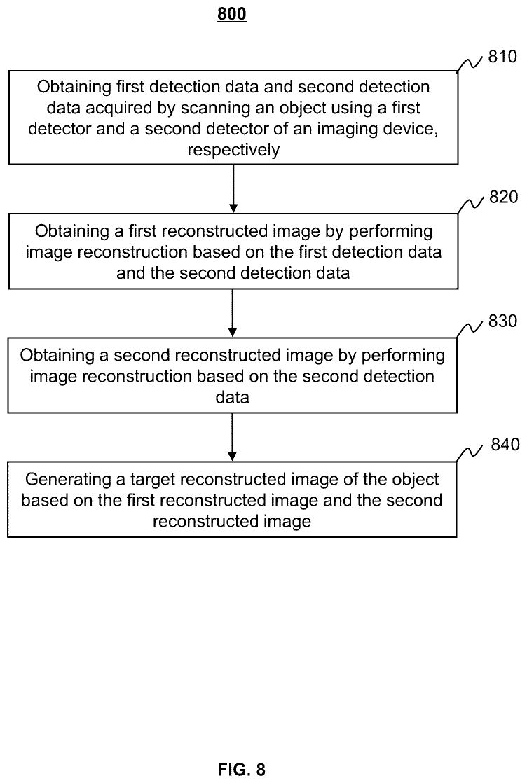

In some embodiments, the processing device may expand the combined data including the first detection data and the virtual detection data based on the second detection data, and obtain third detection data for the additional detection area, thereby extending the scanning FOV of the first detector and obtaining complete detection data corresponding to the first detector. In other words, the composite data of the first detection data, the virtual detection data, and the third detection data corresponds to an area covering the first detection area, the second detection area, and the additional detection area. More descriptions of obtaining the third detection data may be found in FIG. 8 and its related descriptions.

By performing the extension operation on the first detection data corresponding to the first scanning FOV, the data volume of the first detection data can be expanded to be equal with the data volume of the second detection data corresponding to the second scanning FOV, thereby reconstructing a reconstructed image having a same range as the second scanning FOV.

In 620, the target reconstructed image may be obtained based on the second detection data and the extended detection data.

The target reconstructed image may be determined in various ways. In some embodiments, the processing device may reconstruct the second detection data and the extended detection data to obtain a first image and a second image, respectively, and obtain the target reconstructed image based on the first image and the second image.

The first image is an image obtained after reconstructing the second detection data. The second image is an image obtained after reconstructing the extended detection data. The first image and the second image may be CT images.

In some embodiments, a scanning FOV corresponding to the second image may be equal to a scanning FOV corresponding to the second scanning FOV. The second image may be obtained based on the reconstruction of the extended detection data after a data extension operation. During the data extension operation, a difference in data volume may be determined based on a difference between the first scanning FOV and the second scanning FOV. For example, based on the data volume of the first detection data corresponding to the first scanning FOV and the the data volume of the second detection data corresponding to the second scanning FOV, the difference in data volume may be determined. Due to the difference in the scanning FOVs, there may be a difference in the data volume.

In some embodiments, the processing device may reconstruct image(s) based on the second detection data and the extended detection data using various reconstruction techniques. For example, the processing device may perform reconstruction using a filtered inverse projection algorithm, an iterative reconstruction algorithm, an analytical technique, or the like, and the embodiments of the present disclosure do not limit the specific algorithm used for image reconstruction.

Considering that the area in which truncation artifacts typically occur is usually outside the first detection area of a scanning device's detection system (e.g., the area to be extended in the second detection area or the additional detection area). For the dual-source scanning system, the first detection area corresponds to the relatively small scanning FOV (i.e., the first scanning FOV). In other words, the area in which the truncation artifact appears is substantially outside the first scanning FOV and is located in a portion between boundaries of the second scanning FOV. For the second image, an image portion corresponding to the first scanning FOV has relatively complete scanning data collection, and a probability of truncation artifacts is relatively small. Therefore, more attention should be paid to the area to be expanded corresponding to the second scanning FOV and the third probing area when removing the truncation artifacts.

Accordingly, in some embodiments, the processing device may occlude an image area in the second image corresponding to the first scanning FOV, and obtain the target image based on the occluded second image (hereinafter referred to as the third image) and the first image. In some embodiments, the occlusion may be achieved by adding a mask to the image area. In some embodiments, the occluded image area may be a central area of the second image.

In some embodiments, the processing device may determine an image artifact residual based on the third image and the first image, and determine the target image based on the image artifact residual and the second image.

An artifact residual refers to artifact information contained in the second image. The image artifact residual refers to artifact information represented in the form of an image or a matrix.

In some embodiments, the processing device may perform data conversion on the first image and the third image, respectively, to obtain first polar coordinate data and second polar coordinate data, process the first polar coordinate data and the second polar coordinate data to obtain artifact residual data, and perform inverse data conversion on the artifact residual data to obtain the image artifact residual.

Data conversion refers to conversion of data from one format to another format, for example, conversion from data in the form of images to polar coordinate data.

Polar data refers to data that is represented in polar coordinates. In some embodiments, the first polar coordinate data corresponds to a polar coordinate image obtained after converting the first image into polar coordinates. In some embodiments, the second polar coordinate data corresponds to a polar coordinate image obtained after converting the third image into polar coordinates.

In some embodiments, the processing device may convert the first image and the third image to the first polar coordinate data and the second polar coordinate data, respectively, by using a polar coordinate conversion equation or a polar coordinate conversion algorithm.

The artifact residual data may be polarized data. In some embodiments, the processing device may determine the difference between the first polar coordinate data and the second polar coordinate data as the artifact residual data.

In some embodiments, the processing device may perform inverse data conversion on the artifact residual data to obtain the image artifact residual by using a conversion equation or algorithm that is inverse to the conversion equation or algorithm for converting the first image and the third image to the first polar coordinate data and the second polar coordinate data.

The processing device may also determine the image artifact residual in other feasible ways, which are not limited in the present disclosure.

In some embodiments, the processing device may perform calculations to determine the target image based on the second image and the image artifact residual. For example, the processing device may determine the target image by taking a sum of the second image and the image artifact residual.

In some embodiments of the present disclosure, the first image and the second image are reconstructed based on two sets of detection data respectively. By processing the first image and the second image, the artifact residual in the first image, which corresponds to the relatively small scanning FOV, is obtained and removed from the second image, thereby accurately reconstructing the image within the relatively large scanning FOV and improving the accuracy of image reconstruction. Additionally, adding a mask to the area in the second image corresponding to the first scanning FOV and converting the the first image and the third image to the first polar coordinate data and the second polar coordinate data enhances data processing efficiency.

FIG. 7 is a flowchart of an exemplary imaging process according to some other embodiments of the present disclosure. In some embodiments, process 700 may be performed by an electronic device. The electronic device may establish a data connection to the dual-source scanning system described in any embodiment of the present disclosure via a wired or wireless manner, to enable control of an imaging device. As shown in FIG. 7, process 700 may include one or more of the following operations.

In 710, first detection data and second detection data may be acquired by scanning an object using a first detector and a second detector of an imaging device, respectively.

The imaging device refers to a device used to scan an object and generate a scanned image. The object may include a biological object (e.g., a human body, an animal, etc.), a non-biological object (e.g., a body model), etc. In some embodiments, the object may also include a particular portion, organ, and/or tissue of a patient. For example, the object may include the head, chest, legs, etc., or any combination thereof, without limitation herein. In some embodiments, the object may include a specific portion, organ, and/or tissue of the patient and other organs and/or tissues within a certain perimeter thereof.

In some embodiments, the imaging device may be a dual-source Computed Tomography (CT) device. The imaging device described above may also be referred to as a dual-source scanning device. In some embodiments, the dual-source scanning device may be a dual-source flat-scan CT device. In some embodiments, the dual-source scanning device may be a dual-source helical CT device.

In some embodiments, the imaging device may include an asymmetric first detector and a symmetric second detector. In some embodiments, the imaging device may include an asymmetric first detector and an asymmetric second detector.

More descriptions of the first detector and the second detector may be found in FIGS. 1-4B and their related descriptions.

The first detection data refers to detection data obtained when scanning the object using the first detector, and the second detection data refers to detection data obtained when scanning the object using the second detector. Both the first detection data and the second detection data include detection data of the imaging device at a plurality of projection angles. More descriptions of the first detection data and the second detection data may be found in FIG. 5 and its related descriptions.

In 720, a first reconstructed image may be obtained by performing image reconstruction based on the first detection data and the second detection data.

The first reconstructed image is a reconstructed image obtained by image reconstruction based on the first detection data and the second detection data.

The first reconstructed image may be determined in various ways. In some embodiments, the electronic device may reconstruct image(s) based on the second detection data and the first detection data using various reconstruction techniques. For example, the electronic device may perform reconstruction using a filtered inverse projection algorithm, an iterative reconstruction algorithm, an analytical technique, or the like, and the embodiments of the present disclosure do not limit the specific algorithm used for image reconstruction.

In some embodiments, a first scanning FOV of the first detector may include at least a part of a first detection area and at least a part of a second detection area, and a second scanning FOV of the second detector may include at least a part of the first detection area and at least a part of the second detection area. The first detection area is a circular area, the second detection area is a ring area, and the first detection area is surrounded by the second detection area. More descriptions of the first scanning FOV and the second scanning FOV may be found in FIGS. 1-4B and their related descriptions. In some embodiments, ray beams that are emitted from a first radiation source corresponding to the first detector and received by an outermost detector module on one outermost side of the first detector are tangent to the first detection area, and ray beams that are emitted from the first radiation source corresponding to the first detector and received by an outermost detector module on another outermost side of the first detector are tangent to an outer ring of the second detection area, and ray beams that are emitted from a second radiation source corresponding to the second detector and received by outermost detector modules on two outermost sides of the second detector are tangent to or beyond the second detection area. In some embodiments, the first reconstructed image may cover a detection area corresponding to a first scanning FOV (i.e., a sum of the first detection area V1 and a portion of the second detection area V2 shown in FIG. 1) and a detection area corresponding to a second scanning FOV (i.e., a sum of the first detection area V1 and the second detection area V2 shown in FIG. 1).

In some embodiments, if the first detector is a detector with an asymmetric structure, when the first detector is rotated to an arbitrary angle, within the detection area corresponding to the second scanning FOV, an area of the detection area corresponding to the first scanning FOV is larger than an area of the area not covered by the first scanning FOV. Therefore, the electronic device may reconstruct the first reconstructed image covering the detection area corresponding to the second scanning FOV based on the second detection data and the first detection data of the first detector at different positions, thereby expanding an accurate reconstruction range of the imaging.

In some embodiments, the electronic device may obtain extended first detection data by extending the first detection data based on the second detection data, or based on the second detection data and the first detection data, and obtain the first reconstructed image by performing image reconstruction based on the extended first detection data.

In some embodiments, the second scanning FOV of the second detector may include a first detection area and a second detection area. The second detection area may include a detection area corresponding to an extended scanning FOV of the first detector and an area to be expanded. The area to be extended is a part of the detection area that cannot be detected by the first detector (e.g., an area within the second detection area but outside the first scanning FOV of the first detector). In some embodiments, the electronic device may merge a portion of the second detection data corresponding to the area to be extended with the first detection data to extend the first detection data, thereby obtaining extended detection data.

The electronic device may extend the first detection data in a variety of ways. In some embodiments, the electronic device may extend the first detection data based on data from the second detection data that does not overlap with the first detection data.

In some embodiments, the electronic device may determine virtual detection data for the area to be extended based on the first detection data and the second detection data; and obtain the extended first detection databased on the virtual detection data and the first detection data. For example, the electronic device may determine a portion or all of the data from the second detection data that does not overlap with the first detection data as the virtual detection data for the area to be extended. In some embodiments, the area to be extended is an area within the second detection area but outside the first scanning FOV of the first detector.

In some embodiments, the second detection data may include first data and second data, the first data being obtained when a projection angle of the imaging device is at a first preset angle, the second data being obtained when the projection angle of the imaging device is at a second preset angle, the first preset angle and the second preset angle being conjugate. In some embodiments, the first detection data may include third data, the third data being obtained when the projection angle of the imaging device is at the second preset angle. In some embodiments, the first data, the second data and the third data may correspond to the same detection area. In some embodiments, the first data and the second data are conjugated data.

In some embodiments, the electronic device may determine the virtual detection data for the area to be extended based on the first data and difference data between the second data and the third data.

In some embodiments, the virtual detection data may be determined through operations S1-S3:

In S1, first data may be obtained by the electronic device by scanning an object using the second detector when a projection angle of the imaging device is at a first preset angle.

In S2, second data and third data may be obtained by the electronic device by scanning the object using the second detector and the first detector, respectively, when the projection angle of the imaging device is at a second preset angle.

In some embodiments, the first preset angle and the second preset angle are conjugated. In some embodiments, an angular interval between the first preset angle and the second preset angle is a preset interval. The preset interval may be configured such that at the second preset angle, fourth data of the first detector includes the detection data of the area to be extended. For example, the preset interval may be 180° such that the first preset angle and the second preset angle are conjugated.

S3, the virtual detection data for the area to be extended may be determined based on the first data and difference data between the second data and the third data.

The second data and the third data are both detection data of the scanned object, including signal values at various positions of the object. In some embodiments, the difference data of the second data and the third data may be difference data of a same scanning position in the second data and the third data.

In some embodiments, the electronic device may combine the first data with the difference data of the second data and the third data to obtain the virtual detection data for the area to be extended. Merely by way of example, the electronic device may determine the virtual detection data for the area to be extended using Equation (6):

$${I}_{2} = {I}_{1} + ( {I}_{2 p i} - {I}_{1 pi} ) , \qquad ( 6 )$$

wherein I₂ denotes the virtual detection data of the area to be extended, I₁ denotes the first data, I₂ₚᵢ denotes the third data, and I₁ₚᵢ denotes the second data. Each of the virtual detection data, the first data, and the second data includes detection data of different positions of the scanned object. The calculation using Equation (6) refers to calculating the signal values at a same position of the scanned object using Equation (6).

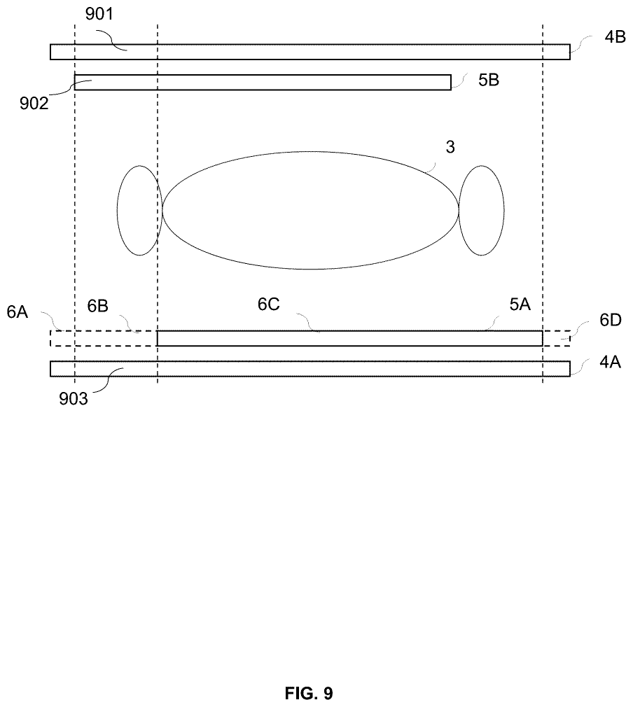

FIG. 9 is a schematic diagram illustrating a determination of exemplary virtual detection data according to some embodiments of the present disclosure. As shown in FIG. 9, 4A and 4B are detection areas in which the second detector is capable of collecting data, 5A and 5B are detection areas in which the first detector is capable of collecting data, 6A and 6D are additional detection areas, 6B is the area to be extended. Data in 4A is the second detection data acquired by the second detector when a projection angle of the imaging device is at a first preset angle, data in 4B is the second detection data acquired by the second detector when the projection angle of the imaging device is at a second preset angle, data in 5A is the first detection data acquired by the first detector when a projection angle of the imaging device is at the first preset angle, data in 5B is the first detection data acquired by the first detector when the projection angle of the imaging device is at the second preset angle, data in 6A and 6D is the extended detection data of the additional detection area, data in 6B is the virtual detection data of the area to be extended, 6C represents a fourth data acquired by the first detector when a projection angle of the imaging device is at the first preset angle. 901 represents the second data acquired by the second detector when the projection angle of the imaging device is at the second preset angle, 902 represents the third data acquired by the first detector when the projection angle of the imaging device is at the second preset angle, 903 represents the first data acquired by the second detector when a projection angle of the imaging device is at the first preset angle. More descriptions of the fourth data may be found in the below descriptions.

The first data 903 and the second data 901 are the second detection data of the second detector, and each of the first data 903 and the second data 901 corresponds to an area covering both the first detection area and the second detection area. The third data 902 and the fourth data 6C are the first detection data of the first detector, and the fourth data 6C is the detection data of an area covered by the first scanning FOV of the first detector. The third data and the fourth data are conjugated data, and the area 5B corresponding to third data covers the area to be extended 6B.

In the above embodiment, the virtual detection data of the area to be extended is determined based on the corresponding scanning positions in the first detection data and the second detection data, and combining the first data, the second data conjugated with the first data, and the third data corresponding to an area covering the area to be extended, thereby improving the accuracy of the obtained virtual detection data.

Since the difference data of the second data and the third data is the difference data of a same scanning position in the second data and the third data, for the same scanning position, the second data and the third data are close to each other. If the difference between the second and third data is large, it indicates an error. In some embodiments, after obtaining the difference data of the second data and the third data, first filtered data may be obtained by filtering data that is greater than or equal to a preset value in the difference data. Then, based on the first filtered data and the first detection data, the virtual detection data for the area to be extended may be determined, which can further improve the accuracy of the obtained virtual detection data. The preset value may be a system default value, an empirical value, a human pre-set value, etc., or any combination thereof, which may be set according to an actual requirement, and the present disclosure does not impose any limitation thereon.

In some embodiments, a low-pass filtering approach (e.g., mean filtering) may be used to filter the data in the difference data that is greater than or equal to the preset value.

In some embodiments, the electronic device may combine the filtering results of the difference data of the second data and the third data, with the first data, to obtain virtual detection data for the area to be extended. Merely by way of example, the electronic device may determine virtual detection data for the area to be extended using Equation (7):

$${I}_{2} = {I}_{1} + Blur ( {I}_{2 p i} - {I}_{1 pi} ) , \qquad ( 7 )$$

wherein Blur denotes a low-pass filtering function.

In some embodiments, the electronic device, after obtaining the first data, the second data, and the third data, may convert each of the first data, the second data, and the third data into data in the form of a parallel beam.

In some embodiments, the electronic device may perform image reconstruction based on the virtual detection data and the first detection data to obtain the first reconstructed image.

In some embodiments, the electronic device may combine the virtual detection data and the first detection data to obtain composite detection data corresponding to an area covering the first detection area and the second detection area, and perform image reconstruction based on the composite detection data to obtain the first reconstructed image with a relatively high degree of accuracy. For example, the electronic device may determine a merging result of the virtual detection data and the first detection data as the composite detection data.

In some embodiments, the first detection data further includes fourth data obtained when the projection angle of the imaging device is at the first preset angle. In some embodiments, the third data and the fourth data are conjugated data.

In some embodiments, the electronic device may further obtain fourth data by scanning the object using the first detector when the projection angle of the imaging device is at the first preset angle. In some embodiments, the electronic device, after obtaining the fourth data, may convert the fourth data into data in the form of a parallel beam.

In some embodiments, the electronic device, after obtaining the virtual detection data, may combine the virtual detection data and the fourth data to obtain composite data corresponding to an area covering the first detection area and the second detection area. Combination methods include splicing, etc. Further, the electronic device may perform image reconstruction based on the composite data to obtain the first reconstructed image. Exemplarily, when the projection angles of the imaging device are at different angles, the electronic device may determine the virtual detection data in the same way described in the above embodiments, determine the composite data corresponding to each angle, and perform image reconstruction based on the composite data corresponding to the angles to obtain the first reconstructed image.

It should be noted that the electronic device may perform image reconstruction based on the first detection data and the second detection data. Alternatively, the electronic device may send the detection data (e.g., the first detection data or the second detection data) to a third-party device (e.g., a server for reconstructing a CT image) in order to facilitate the third-party device to perform image reconstruction based on the detection data.

In some embodiments of the present disclosure, the virtual detection data of the area to be extended is determined based on the first detection data and the second detection data, thereby extending the first detection data of the first detector, and the extended first detection data is the detection data corresponding to an area covering the first detection area and the second detection area. Further, a first reconstructed image with a relatively high degree of accuracy can be reconstructed based on the detection data corresponding to the area covering the first detection area and the second detection area.

In some embodiments, after obtaining the first reconstructed image, the electronic device may perform image reconstruction based on the second detection data to obtain a second reconstructed image. Subsequently, the electronic device may generate a target reconstructed image based on the first reconstructed image and the second reconstructed image. See FIG. 7 and its related description for a more detailed description of this embodiment.

FIG. 8 is a flowchart of an exemplary imaging process according to some other embodiments of the present disclosure. In some embodiments, process 700 may be performed by an electronic device. The electronic device may establish a data connection to the dual-source scanning system described in any embodiment of the present disclosure via a wired or wireless manner, to enable control of an imaging device. As shown in FIG. 8, process 800 may include one or more of the following operations.

In 810, first detection data and second detection data may be obtained by scanning a same object using a first detector and a second detector on the imaging device, respectively.

See operation 710 and its related description for more descriptions of obtaining the first detection data and the second detection data.

In 820, a first reconstructed image may be obtained by performing image reconstruction based on the first detection data and the second detection data.

See operation 720 and its related description for more descriptions of obtaining the first reconstructed image.

In 830, a second reconstructed image may be obtained by performing image reconstruction based on the second detection data.

The second reconstructed image is a reconstructed image obtained by image reconstruction based on the second detection data.

In some embodiments, the electronic device may reconstruct the second detection data using various reconstruction techniques. For example, the electronic device may perform reconstruction using a filtered inverse projection algorithm, an iterative reconstruction algorithm, an analytical reconstruction algorithm, etc., and the embodiments of the present disclosure do not limit the specific algorithm used for image reconstruction.

In 840, a target reconstructed image of the object may be generated based on the first reconstructed image and the second reconstructed image.

In some embodiments, the electronic device may synthesize the first reconstructed image and the second reconstructed image to obtain the target reconstructed image.

In some embodiments, the first reconstructed image may be obtained based on the first detection data collected by the first detector when scanning the object using a first energy spectrum, and the second reconstructed image may be obtained based on the second detection data collected by the first detector when scanning the object using a second energy spectrum. In other words, the first reconstructed image may be a reconstructed image of a first energy (e.g., a CT image of the first energy) and the second reconstructed image is a reconstructed image of a second energy (e.g., a CT image of the second energy). Therefore, synthesizing the first reconstructed image and the second reconstructed image enables synthesis of reconstructed images of different energies to obtain a quantitative target reconstructed image. The synthesis of reconstructed images of different energies refers to imaging based on the characteristic that different substances have different attenuation coefficient variations at different energies. The target reconstructed image obtained by the above embodiments can be generated based on reconstructed images of different energies, and a composition ratio of the scanned object can be accurately obtained.

In some embodiments, a preset image synthesis model may be used to synthesize reconstructed images of different energies. Merely by way of example, the image synthesis model may take the form of one or more of a GAN-based approach, a diffusion model approach, an autoregressive approach, a neural radiation field (NeRF) approach, or the like. The embodiments of the present disclosure do not have a special limitation on the image synthesis model, and it is sufficient to adopt operations known to those skilled in the art.

In some embodiments, the second scanning FOV of the second detector may further include an additional detection area located peripheral to the second detection area. Correspondingly, the second detection data of the second detector corresponds to an area covering the third detection area, the second reconstructed image obtained based on the second detection data may correspond to an area covering the additional detection area, and the above first detection data and the virtual detection data may correspond to an area covering the first detection area and the second detection area.

In some embodiments, the electronic device may extend the composite detection data including the first detection data and the virtual detection data based on the second detection data, and obtain third detection data for the additional detection area, thereby expanding the scanning FOV of the first detector and obtaining complete detection data corresponding to the first detector, i.e., the first detection data, the virtual detection data, and the third detection data are the detection data of the first detection area, the second detection area, and the additional detection area. For example, the third detection data may be obtained by deriving detection data for the additional detection area corresponding to the first detector based on the second detection data. The above derivation process is the same as the data derivation process for the dual-source scanning device with two symmetrical detectors and will not be repeated here.

In some embodiments, the electronic device may reconstruct a third reconstructed image based on the first detection data, the virtual detection data, and the third detection data. The third reconstructed image may correspond to an area covering the first detection area, the second detection area, and the additional detection area. The electronic device may then generate, based on the second reconstructed image and the third reconstructed image, a synthetic reconstructed image of the scanned object located within an area including the additional detection area, the second detection area, and the first detection area.

In some embodiments, the second reconstructed image and the third reconstructed image are reconstructed images that are different in energy, but have a structural similarity. After obtaining the second reconstructed image and the third reconstructed image, the electronic device may correct an artifact of the third reconstructed image based on the similarity between the second reconstructed image and the third reconstructed image to obtain a corrected image, and then generate the synthetic reconstructed image based on the first reconstructed image and the corrected image.

In some embodiments, the electronic device may obtain the synthetic reconstructed image directly based on the first reconstructed image and the third reconstructed image.

For example, when the object scanned by a dual-source scanning device is pure water, the detectors with different energy spectra may have the same detection data and reconstruction values. When the scanned object includes pure water and other substances, an error in the reconstructed image may be determined based online integration path differences and line attenuation coefficient differences of the pure water and the substances.

If the first reconstructed image is obtained based on the method provided in some embodiments of the present disclosure, an error ΔI_{pi+I1} may be determined according to Equation (8):

$$Δ {I}_{pi + I 1} \approx {I}_{{2}_{p i}} + ( {I}_{1} - {I}_{{1}_{p i}} ) - {I}_{2} \approx {μ}_{2}^{H 2 O} {l}_{p i}^{H 2 O} + {μ}_{2}^{B} {l}_{p i}^{B} + {μ}_{1}^{H 2 O} {l}^{H 2 O} + {μ}_{1}^{B} {l}^{B} - {μ}_{1}^{H 2 O} {l}_{p i}^{H 2 O} - {μ}_{1}^{B} {l}_{p i}^{B} - {μ}_{2}^{H 2 O} {l}^{H 2 O} - {μ}_{2}^{B} {l}^{B} \approx ( {μ}_{2}^{B} - {μ}_{1}^{B} ) ( {l}^{B} - {l}_{p i}^{B} ) . \qquad ( 8 )$$

If the first reconstructed image is obtained base on the second detection data of the second detector only, an error ΔIₚᵢ may be determined using Equation (9):

$$Δ {I}_{I 1} \approx {I}_{1} - {I}_{2} \approx {μ}_{1}^{H 2 O} {l}^{H 2 O} + {μ}_{1}^{B} {l}^{B} - {μ}_{2}^{H 2 O} {l}^{H 2 O} - {μ}_{2}^{B} {l}^{B} \approx ( {μ}_{2}^{B} - {μ}_{1}^{B} ) {l}^{B} . \qquad ( 9 )$$

If the first reconstructed image is obtained base on the detection data collected by the first detector scanning the object at a plurality of angles, an error ΔI_{I1}, may be determined according to Equation (10):

$$Δ {I}_{p i} \approx {I}_{{2}_{p i}} - {I}_{2} \approx {μ}_{2}^{H 2 O} {l}_{p i}^{H 2 O} + {μ}_{2}^{B} {l}_{p i}^{B} - {μ}_{2}^{H 2 O} {l}^{H 2 O} - {μ}_{2}^{B} {l}^{B} \approx {μ}_{2}^{H 2 O} ( {l}_{p i}^{H 2 O} - {l}^{H 2 O} ) + {μ}_{2}^{B} ( {l}_{p i}^{B} - {l}^{B} ) , \qquad ( 10 )$$

wherein μ₁^{H2O} denotes a line attenuation coefficient difference of the pure water detected by the first detector, μ₁^{B} denotes a line attenuation coefficient difference of the other substance detected by the first detector, μ₂^{H2O} denotes a line attenuation coefficient difference of the pure water detected by the second detector, μ₂^{B} denotes a line attenuation coefficient difference of the other substance detected by the second detector; l^{H2O} denotes a line integration path difference of the pure water, lₚᵢ^{H2O} denotes a line integration path difference of the dual-source scanning device rotated by 180°, l^{B} denotes a line integration path difference of the other substance, and lₚᵢ^{B} denotes a line integration path difference of the other substance after the dual-source scanning device is rotated by 180°.

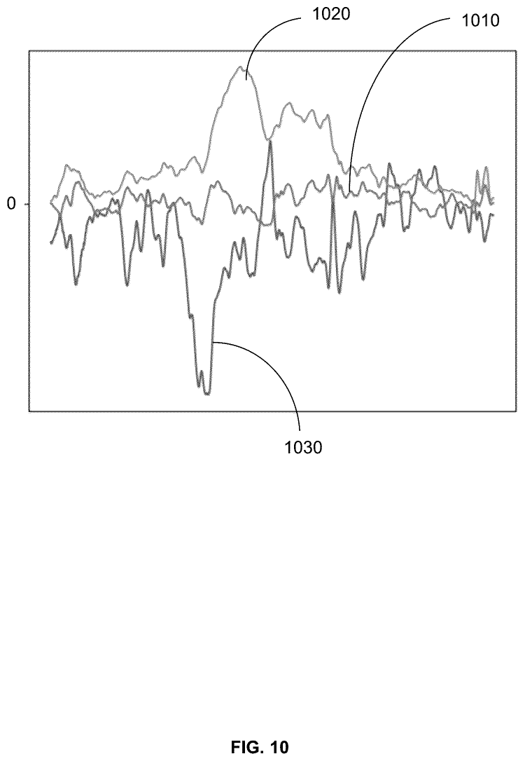

FIG. 10 is a schematic diagram illustrating comparison of errors in a first reconstructed image obtained using an imaging method according to some embodiments of the present disclosure. As shown in FIG. 10, the horizontal coordinate represents the pixel position, the vertical coordinate represents the error, curve 1010 represents the error ΔI_{pi+I1}, curve 1020 represents the error ΔI_{I1}, and curve 1030 represents the error ΔIₚᵢ, and it may be seen that the error ΔI_{pi+I1} is close to 0. Thus, using the imaging method provided in some embodiments of the present disclosure, a first reconstructed image with higher accuracy can be reconstructed, which in turn improves the accuracy of the target reconstructed image of the scanned object obtained.

It should be understood that the serial numbers of the operations in the above embodiments do not imply the order of execution, and the order of execution of the processes should be determined by their functions and internal logic without constituting any limitation of the process of implementing the embodiments of the present disclosure.

Since each of the first reconstructed image and the second reconstructed image cover the complete second detector area, the accuracy of data in the target reconstructed image corresponding to the area that cannot be covered by the scanning FOV of the first detector can be improved, an accurate reconstruction range of the reconstructed image can be enlarged, and then a target reconstructed image with a high degree of accuracy can be obtained.

In some embodiments, after obtaining the first reconstructed image and the second reconstructed image, the electronic device may correct the artifact in the first reconstructed image based on the second reconstructed image, i.e., to remove the artifact in a portion of the first reconstructed image corresponding to the area to be extended, thereby obtaining a corrected first reconstructed image with an accuracy higher than the accuracy of the first reconstructed image.

Since the second reconstructed image is determined based on the second detection data and the first reconstructed image is determined based on the first detection data, the first reconstructed image and the second reconstructed image are reconstructed images of different energies. However, since both the first and second reconstructed images are reconstructed images of a same scanned object, there is a structural similarity between the first and second reconstructed images, and the second reconstructed image with a relatively low energy spectrum has a relatively high contrast. The electronic device may determine the similarity between the second reconstructed image and the first reconstructed image, thereby determining difference information between the first reconstructed image and the second reconstructed image. Further, the electronic device may determine the artifact in the first reconstructed image based on the difference information (e.g., by determining an area in the first reconstructed image with a large difference in similarity as the artifact), correct the data in the area, and obtain a corrected first reconstructed image with a higher accuracy, thereby improving the accuracy of the target reconstructed image obtained subsequently.

In some embodiments, after obtaining the target reconstructed image of the object based on the first reconstructed image and the second reconstructed image, the electronic device may obtain a corrected first reconstructed image with an accuracy higher than the accuracy of the first reconstructed image by correcting the first reconstructed image based on the target reconstructed image and the first detection data, and generate a corrected target reconstructed image of the object based on the second reconstructed image and the corrected first reconstructed image.

In some embodiments, after obtaining the target reconstructed image, the electronic device may correct the virtual detection data based on the target reconstructed image to obtain corrected virtual detection data. Corrected extended detection data that include the corrected virtual detection data and the first detection data may be obtained, which can improve the accuracy of the extended detection data. Subsequently, a corrected first reconstructed image having an accuracy higher than the accuracy of the first reconstructed image may be obtained based on the corrected extended detection data.

In some embodiments, the virtual detection data may be corrected based on the target reconstructed image by: determining detection data corresponding to an area to be extended based on the target reconstructed image, correcting an artifact in the virtual detection data based on the detection data corresponding to the area to be extended, and obtaining the corrected virtual detection data.

Since the target reconstructed image is obtained based on the second reconstructed image, the accuracy of the portion of the target reconstructed image corresponding to the area to be extended is higher. The target reconstructed image includes detection data for each position of the scanned object, the electronic device may determine detection data for the area to be expanded based on the target reconstructed image, correct the artifact in the virtual detection data based on difference information between the detection data for the area to be expanded and the virtual detection data, and obtain the corrected virtual detection data.

In some embodiments, the electronic device may employ a forward projection correction technique to correct the artifact in the virtual detection data. For example, the electronic device may perform forward projection on the target reconstructed image to obtain a line integral path difference, and determine the difference information between the detection data of the area to be extended and the virtual detection data based on the line integral path difference and a line attenuation coefficient difference. The line integral path difference and the line attenuation coefficient difference are related to the composition of the scanned object. After obtaining the difference information, the electronic device may correct the virtual detection data based on the difference information, making the corrected virtual detection data approximate the detection data of the area to be extended. For example, the difference information may include an error corresponding to each of a plurality of positions in the area to be extended, and the virtual detection data corresponding to the plurality of positions minus the errors corresponding to the plurality of positions may be determined as the corrected virtual detection data.

In the above embodiments, by correcting the artifact in the virtual detection data and obtaining the target reconstructed image based on the corrected virtual detection data and the first detection data, the accuracy of the obtained target reconstructed image can be improved.

In some embodiments, the electronic device may determine a structural similarity between the first reconstructed image and the second reconstructed image; and generate a corrected target reconstructed image of the object by correcting the first reconstructed image based on the structural similarity between the first reconstructed image and the first reconstructed image.

Since the first reconstructed image and the second reconstructed image are reconstructed images of different energies, the first reconstructed image and the second reconstructed image have structural similarity.

In some embodiments, the electronic device may compare the similarity between the brightness and contrast of the first reconstructed image and the second reconstructed image to determine the structural similarity between the first reconstructed image and the second reconstructed image.

In some embodiments, the electronic device may correct an artifact of the the first reconstructed image based on the structural similarity between the first reconstructed image and the second reconstructed image to obtain a corrected first reconstructed image, and then generate the corrected target reconstructed image based on the corrected first reconstructed image and the second reconstructed image.

In the above embodiments, by correcting the artifact in the first reconstructed image and obtaining the target reconstructed image based on the corrected first reconstructed image and the second reconstructed image, the accuracy of the obtained target reconstructed image can be improved.

It is to be noted that the information interaction, execution process, etc., between the above devices/units are based on the same concept as the method embodiments of the present disclosure, the specific functions and technical effects brought about by them may be specifically referred to in the section of method embodiments, which will not be repeated herein.

Some embodiments of the present disclosure further provide a method implemented on at least one machine each of which has at least one processor and a storage device for controlling a dual-source scanning system, wherein the dual-source scanning system includes an asymmetric first detector and a symmetric second detector, the asymmetric first detector has a first scanning field of view (FOV), the symmetric second detector has a second scanning FOV, and the first scanning FOV is smaller than the second scanning FOV, the method comprising: determining a part of an object to be scanned; obtaining first detection data and second detection data by controlling the asymmetric first detector and the second detector to scan the part to be scanned, respectively; and obtaining a target reconstructed image by performing image reconstruction based on the first detection data and the second detection data, wherein a coverage corresponding to the first reconstructed image includes the first detection area and the second detection area.

More descriptions of image reconstruction may be found in the related descriptions in FIGS. 6-9.

Some embodiments of the present disclosure further provide a dual-source helical scanning system. The dual-source helical scanning system may include a first detector and a second detector, wherein: at least one of the first detector or the second detector has an asymmetric structure relative to a central channel thereof, and a first scanning field of view (FOV) of the first detector and a second scanning FOV of the second detector are at least partially overlapped. In some embodiments, the first detector of the dual-source helical scanning system has an asymmetric structure relative to a first central channel of the first detector, a scan pitch of the dual-source helical scanning system may be linked to a first scanning FOV of the first detector.

By setting the scan pitch of the dual-source helical scanning system during scanning, a range of the first scanning FOV may be adjusted to a certain extent. In other words, to improve data sufficiency, it is necessary to expand the first scanning FOV as much as possible, and the pitch needs to satisfy a threshold condition. When the pitch satisfy the threshold condition, the dual-source helical scanning system can satisfy a data sufficiency condition for an area to be expanded after the asymmetrical first detector is extended. For example, the pitch needs to satisfy the threshold condition shown in Equation (4). For more descriptions of Equation (4), see operation 620 and its related descriptions.

In some embodiments, the scan pitch of the dual-source helical scanning system may be linked with the first scanning FOV of the first detector, including: a maximum pitch of the dual-source helical scanning system may be related to at least one of a distance from a focal point of the first detector to a isocenter of the dual-source scanning system, a distance from any point in the first scanning FOV to the isocenter, or a count of layers of detector modules in a movement direction of a couch of the dual-source helical scanning system. More descriptions of this embodiment may be found in operation 720 and its related descriptions.

In addition, for the dual-source helical scanning system, during fast scanning with an ultra-large pitch, relying solely on a detector system with a relatively large scanning FOV for scanning may result in missing data. In this case, simultaneous scanning with a detector system with a relatively small scanning FOV may compensate for the missing data in a scanning trajectory. However, there may still be data insufficiency when performing reconstruction on an area outside the relatively small scanning FOV. In this case, the problem of data insufficiency may be alleviated through simple extrapolation.

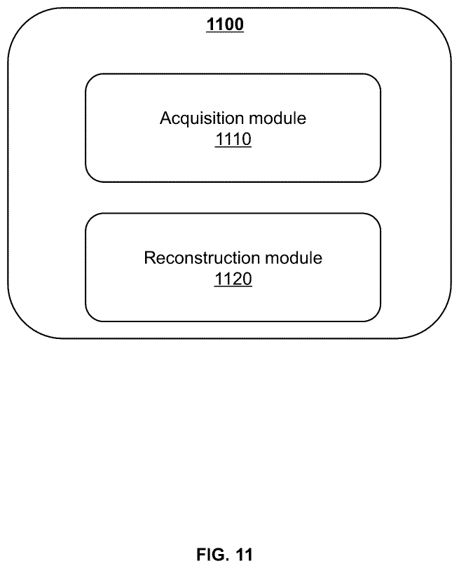

Some embodiments of the present disclosure further provide an imaging device. FIG. 11 is a schematic diagram of an exemplary imaging device according to some embodiments of the present disclosure. As shown in FIG. 11, an imaging device 1100 includes an acquisition module 1110 and a reconstruction module 1120.

In some embodiments, the acquisition module 1110 may be configured to obtain first detection data and the second detection data acquired by scanning an object using a first detector and a second detector of an imaging device, respectively.

In some embodiments, the reconstruction module 1120 may be configured to obtain a first reconstructed image by performing image reconstruction based on the first detection data and the second detection data.

More descriptions of obtaining the first detection data and the second detection data and the image reconstruction may be found in the related descriptions in FIGS. 6-10.

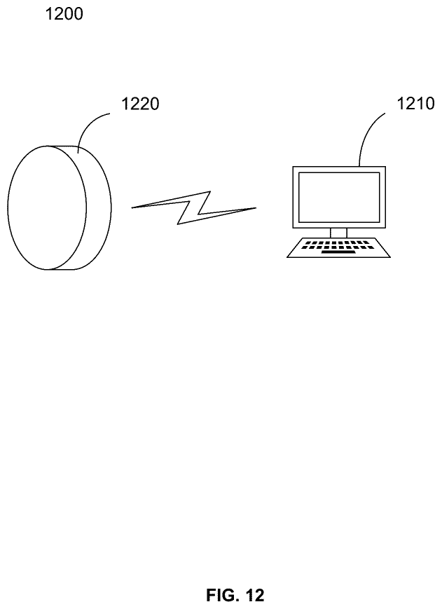

Some embodiments of the present disclosure further provide an imaging system. FIG. 12 is a schematic diagram of an exemplary imaging system according to some embodiments of the present disclosure. As shown in FIG. 12, an imaging system 1200 includes an electronic device 1210 and an imaging device 1220. The imaging method provided in some embodiments of the present disclosure may be applied to the imaging system 1200 as shown in FIG. 12.

The electronic device 1210 and the imaging device 1220 may establish a communication connection directly or may communicate with each other through a server, which is not limited by the embodiments of the present disclosure.

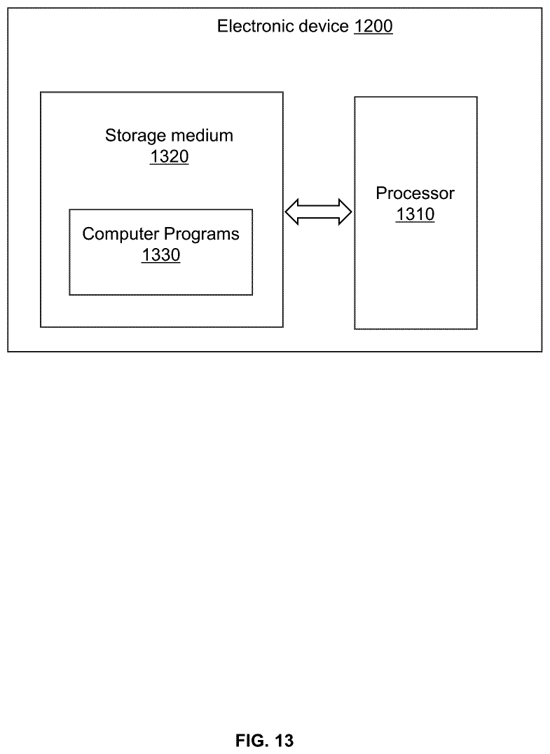

Merely by way of example, the electronic device 1210 may be a computing device such as a desktop computer, a laptop, a PDA, etc. The electronic device 1210 may also be an imaging device or a device that includes an imaging device, in which case the imaging device 1220 shown in FIG. 12 may be omitted. It should be noted that if the electronic device 1210 is an imaging device, the electronic device 1210 not only has a function of reconstructing an image, but also has a function of an imaging device. For example, the electronic device 1210 may have a processing module and a scanning module, wherein the processing module may be configured to implement the method provided by embodiments of the present disclosure. The scanning module may include a first detector and a second detector, wherein a scanning FOV of the first detector may be smaller than a scanning FOV of the second detector, and field angles on two sides of a central channel of the first detector may be unequal. See FIG. 13 and its related description for a more detailed description of the electronic device.

Some embodiments of the present disclosure further provide an electronic device. FIG. 13 is a schematic diagram of an exemplary electronic device according to some embodiments of the present disclosure. As shown in FIG. 13, an electronic device 1200 may include at least one processor 1310 and at least one storage medium 1320. The at least one storage medium 1320 may store computer programs 1330, and the at least one processor 1310 may be configured to execute at least a portion of the computer programs 1330 to implement the imaging method as described in any of the embodiments of the present disclosure, for example, operations 710-720 shown in FIG. 7. Alternatively, the at least one processor 1310 may achieve the functions of each module/unit in each of the above device embodiments (e.g., the functions of the acquisition module 1110 and the reconstruction module 1120 shown in FIG. 11) when executing the computer programs 1330.

Exemplarily, the computer programs 1330 may be partitioned into one or more modules/units, the one or more modules/units being stored in the at least one storage medium 1320 and executed by the at least one processor 1310 to implement the imaging method described in any one of the embodiments of the present disclosure. The one or more of the modules/units may be a series of computer program instruction segments capable of accomplishing a particular function, which are used to describe the process of execution of the computer programs 1330 in the electronic device 1200.

It may be understood by those skilled in the art that FIG. 13 merely provides an exemplary electronic device and does not constitute a limitation of the electronic device, which may include more or fewer components than illustrated, or a combination of some components described in FIG. 13, or different components not described in FIG. 13. For example, the described electronic device may also include an input/output device, a network access device, a bus, or the like.

In some embodiments, the at least one processor 1310 may include at least one of a Central Processing Unit (CPU), a Digital Signal Processor (DSP), an Application Specific Integrated Circuit (ASIC), a Field-Programmable Gate Array (FPGA), a general-purpose processor, a programmable logic device, a discrete gate or transistor logic device, a discrete hardware component, or the like. The general purpose processor may be a microprocessor or any conventional processor, etc.

In some embodiments, the at least one storage medium 1320 may be an internal storage unit of the electronic device 1200, such as a hard disk or memory of the electronic device 1200. The at least one storage medium 1320 may also be an external storage device of the electronic device 1200, such as a plug-in hard disk equipped on the electronic device 1200, a Smart Media Card (SMC), a Secure Digital (SD) card, a flash card, or the like. Further, the at least one storage medium 1320 may also include both an internal storage unit and an external storage device of the electronic device 1200. In some embodiments, the at least one storage medium 1320 may be configured to store the computer programs 1330 as well as other programs and data required by the electronic device 1200. In some embodiments, the at least one storage medium 1320 may be configured to temporarily store data that has been output or will be output.

A person of ordinary skill in the art may clearly understand that, for the convenience and conciseness of the description, only the division of each functional unit and module described above is given as an example, and that, in actual application, the functions described above may be accomplished by different functional units and modules according to needs, i.e., the internal structure of the device described above is divided into different functional units or modules for accomplishing all or some of the above-described functions. The various functional units and modules in the embodiments may be integrated in a single processing unit, or different units may physically exist separately, or two or more units may be integrated in a single unit, and the above integrated units may be realized either in the form of hardware or in the form of software functional units. In addition, the specific names of the functional units and modules are only for distinguishing purposes and are not intended to limit the scope of protection of the present disclosure. The specific working process of the units and modules in the above system may be referred to the corresponding process in the foregoing embodiments of the method, and will not be repeated herein.

In the above embodiments, the description of each embodiment focus on different aspects. Parts not detailed or recorded in one embodiment may be referenced in the relevant descriptions of other embodiments.

In the embodiments provided in the present disclosure, it should be understood that the disclosed device/electronic device and method may be implemented in other ways. For example, the device/electronic device described in the above embodiments are merely illustrative. For example, the division of the described modules or units is merely a logical functional division, and the modules or units may be divided in another way in actual implementation, e.g., a plurality of units or components may be combined or may be integrated into another system, or some features may be ignored, or not implemented. Moreover, the coupling or direct coupling or communication connection shown or discussed in the present disclosure may be indirect coupling or communication connection through an interface, a device, or a unit, and may be in electrical, mechanical, or other forms.

The units described as separate components may be, but are not necessarily, physically separated. The components shown as units may be, but are not necessarily, physical units. In other words, the components may be located in one place or distributed across multiple network units. Depending on actual needs, some or all of the units may be selected to achieve the objectives of the embodiments.

If the integrated modules/units are implemented as software functional units and sold or used as independent products, they may be stored in a computer-readable storage medium. Based on this understanding, all or part of the processes in the above-described method embodiments may also be carried out by a computer program instructing the relevant hardware to complete. The computer program may be stored in a computer-readable storage medium, and when executed by a processor, the computer program may implement the operations of the method embodiments described above. The computer program may include a computer program code, which may be in the form of source code, in the form of object code, in the form of an executable file or in some intermediate form, or the like. The computer-readable medium may include: any entity or device capable of carrying the computer program code, a recording medium, a USB flash drive, a removable hard disk, a diskette, a CD-ROM, a computer memory, a Read-Only Memory (ROM), a Random Access Memory (RAM), an electric carrier signal, a telecommunication signal, a software distribution medium, or the like.

A person of ordinary skill in the art may realize that the units and algorithmic operations of the various examples described in conjunction with the embodiments disclosed herein are capable of being implemented in electronic hardware, or a combination of computer software and electronic hardware. Whether these functions are performed in hardware or software depends on the specific application and design constraints of the technical solution. The skilled professional may use different methods to implement the described functions for each specific application, but such implementations should not be considered beyond the scope of the present disclosure.

The above-mentioned embodiments are only intended to illustrate the technical solutions of the present disclosure, and not to limit them. Although the present disclosure has been described in detail with reference to the aforementioned embodiments, those skilled in the art should understand that they can still modify the technical solutions described in the foregoing embodiments or make equivalent replacements for some of the technical features; such modifications or replacements do not depart from the scope of the technical solutions of the embodiments of the present disclosure and should be included within the protection scope of the present disclosure.

Having thus described the basic concepts, it may be rather apparent to those skilled in the art after reading this detailed disclosure that the foregoing detailed disclosure is intended to be presented as illustrative example and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

As another example, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This way of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameter set forth in the written description and attached claims are approximations that may vary depending upon the desired properties sought to be obtained by a particular embodiment. In some embodiments, the numerical parameter should be construed in light of the number of reported significant digits and by applying ordinary rounding techniques. Notwithstanding that the numerical ranges and parameter setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrating of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A method implemented on at least one machine each of which has at least one processor and a storage device for imaging, the method comprising:
obtaining first detection data and second detection data acquired by scanning an object using a first detector and a second detector of an imaging device respectively, wherein field angles on two sides of a central channel of the first detector are unequal, and a first scanning FOV of the first detector and a second scanning FOV of the second detector are at least partially overlapped; and
obtaining a first reconstructed image by performing image reconstruction based on the first detection data and the second detection data.

2. The method of claim 1, wherein:
the first scanning FOV of the first detector includes at least a part of a first detection area and at least a part of a second detection area,
the second scanning FOV of the second detector includes at least a part of the first detection area and at least a part of the second detection area,
the first detection area is a circular area, the second detection area is an annular area, and the first detection area being surrounded by the second detection area;
ray beams that are emitted from a first radiation source corresponding to the first detector and received by an outermost detector module on one outermost side of the first detector are tangent to the first detection area,
ray beams that are emitted from the first radiation source corresponding to the first detector and received by an outermost detector module on another outermost side of the first detector are tangent to an outer ring of the second detection area;
ray beams that are emitted from a second radiation source corresponding to the second detector and received by outermost detector modules on two outermost sides of the second detector are tangent to or beyond the second detection area; and
a coverage corresponding to the first reconstructed image includes the first detection area and the second detection area.

3. The method of claim 2, wherein the obtaining a first reconstructed image by performing image reconstruction based on the first detection data and the second detection data includes:
obtaining extended first detection data by extending the first detection data based on the second detection data, or based on the second detection data and the first detection data; and
obtaining the first reconstructed image by performing image reconstruction based on the extended first detection data.

4. The method of claim 3, wherein the obtaining extended first detection data by extending the first detection data based on the second detection data and the first detection data includes:
determining virtual detection data for an area to be extended based on the first detection data and the second detection data, wherein the area to be extended is an area detected by the second detector but not detected by the first detector; and
obtaining the extended first detection data based on the virtual detection data and the first detection data.

5. The method of claim 4, wherein the second detection data includes first data and second data, the first data being obtained when a projection angle of the imaging device is at a first preset angle, the second data being obtained when the projection angle of the imaging device is at a second preset angle, the first preset angle and the second preset angle being conjugate; and the first detection data includes third data, the third data being obtained when the projection angle of the imaging device is at the second preset angle;
the determining virtual detection data for an area to be extended based on the first detection data and the second detection data includes:
determining the virtual detection data for the area to be extended based on the first data and difference data between the second data and the third data.

6. The method of claim 4, wherein the first detection data further includes fourth data obtained when the projection angle of the imaging device is at the first preset angle, and the obtaining the first reconstructed image by performing image reconstruction based on the virtual detection data and the first detection data includes:
generating composite data corresponding to an area covering the second detection area by splicing the virtual detection data and the fourth data; and
obtaining the first reconstructed image by performing image reconstruction based on the composite data.

7. The method of claim 2, further comprising:
obtaining a second reconstructed image by performing image reconstruction based on the second detection data; and
generating a target reconstructed image of the object based on the first reconstructed image and the second reconstructed image.

8. The method of claim 7, further comprising:
obtaining a corrected first reconstructed image by correcting the first reconstructed image based on the target reconstructed image and the first detection data; and
generating a corrected target reconstructed image of the object based on the second reconstructed image and the corrected first reconstructed image.

9. The method of claim 7, further comprising:
determining a structural similarity between the first reconstructed image and the second reconstructed image; and
generating a corrected target reconstructed image of the object by correcting the first reconstructed image based on the structural similarity between the first reconstructed image and the second reconstructed image.

10. A dual-source scanning system, comprising a first detector and a second detector, wherein:
at least one of the first detector or the second detector has an asymmetric structure relative to a central channel thereof, and
a first scanning field of view (FOV) of the first detector and a second scanning FOV of the second detector are at least partially overlapped.

11. The system of claim 10, wherein:
the first detector has an asymmetric structure relative to a first central channel of the first detector,
the first scanning FOV of the first detector includes a first detection area and at least a part of a second detection area,
the second scanning FOV of the second detector includes the first detection area and the second detection area,
the first detection area is surrounded by the second detection area, and
the first scanning FOV of the first detector is smaller than the second scanning FOV of the second detector.

12. The system of claim 10, wherein the first detector has an asymmetric structure relative to a first central channel of the first detector, the second detector has an asymmetric structure relative to a second central channel of the second detector,
the first scanning FOV of the first detector includes at least a part of a first detection area and at least a part of a second detection area, and the second scanning FOV of the second detector includes at least a part of the first detection area and at least a part of the second detection area, the first detection area being surrounded by the second detection area; or
the first scanning FOV of the first detector includes at least a part of a third detection area, and the second scanning FOV of the second detector includes at least a part of a fourth detection area, the third detection area and the fourth detection area being partially overlapped.

13. The system of claim 10, wherein the asymmetric structure is configured as counts of detector modules on two sides relative to the central channel being different.

14. The system of claim 10, wherein the asymmetric structure is configured as arrangement curvatures of detector modules on two sides relative to the central channel being different.

15. The system of claim 11, wherein the asymmetric structure of the first detector is configured as:
a count of detector modules on a side close to a second focal point relative to the first central channel of the first detector being greater than a count of detector modules on a side away from the second focal point relative to the first central channel of the first detector, the second focal point being the focal point of the second detector.

16. The system of claim 12, wherein:
the asymmetric structure of the first detector is configured as: a count of detector modules on a side close to a second focal point relative to the first central channel of the first detector being greater than a count of detector modules on a side away from the second focal point relative to the first central channel of the first detector, the second focal point being the focal point of the second detector; and
the asymmetric structure of the second detector is configured as: a count of detector modules on a side close to a first focal point relative to the second central channel of the second detector being greater than a count of detector modules on a side away from the first focal point relative to the second central channel of the second detector, the first focal point being the focal point of the first detector.

17. The system of claim 10, wherein:
a scan pitch of the dual-source scanning system is linked to the first scanning FOV of the first detector, and
a maximum pitch of the dual-source scanning system is related to at least one of a distance from a focal point of the first detector to the isocenter, a distance from any point in the first scanning FOV to the isocenter, or a count of detector modules of the first detector in a movement direction of a couch of the dual-source scanning system.

18. A method implemented on at least one machine each of which has at least one processor and a storage device for controlling a dual-source scanning system, wherein the dual-source scanning system includes a first detector and a second detector, at least one of the first detector and the second detector has an asymmetric structure relative to a central channel thereof, the first detector has a first scanning field of view (FOV), the second detector has a second scanning FOV, and the first scanning FOV and the second scanning FOV are at least partially overlapped, the method comprising:
obtaining first detection data acquired by the first detector and second detection data acquired by the second detector by scanning a part of an object to be scanned; and
generating a target reconstructed image by performing image reconstruction based on the first detection data and the second detection data.

19. The method of claim 18, wherein the first scanning FOV includes a first detection area and a part of a second detection area, and the second scanning FOV includes the first detection area and the second detection area, the first detection area being surrounded by the second detection area;
the generating a target reconstructed image by performing image reconstruction based on the first detection data and the second detection data includes:
obtaining extended detection data by performing an extension operation on the first detection data, a scanning FOV corresponding to the extended detection data being larger than the first scanning FOV; and
obtaining the target reconstructed image based on the second detection data and the extended detection data.

20. The method of claim 17, wherein the scanning a part of an object to be scanned includes:
scanning the part of the object to be scanned using a helical scan;
wherein a maximum pitch of the helical scan is related to at least one of a distance from a focal point of the first detector to an isocenter of the dual-source scanning system, a distance from any point in the first scanning FOV to the isocenter, or a count of detector modules of the first detector in the movement direction of the couch.
